(19) United States
(12) Patent Application Publication
(10) Pub. No.: US 2017/0014446 A1
(43) Pub. Date: Jan. 19, 2017

(54) COMPOSITIONS AND METHODS FOR ADMINISTERING GALECTIN ANTAGONISTS

(71) Applicant: LA JOLLA PHARMACEUTICAL COMPANY, San Diego, CA (US)

(72) Inventors: James Rolke, San Diego, CA (US); George Tidmarsh, Portola Valley, CA (US)

(73) Assignee: LA JOLLA PHARMACEUTICAL COMPANY, San Diego, CA (US)

(21) Appl. No.: 15/125,051

(22) PCT Filed: Mar. 10, 2015
(86) PCT No.: PCT/US15/19691

§ 371 Date: Sep. 9, 2016

Related U.S. Application Data
(60) Provisional application No. 61/950,806, filed on Mar. 10, 2014.

(51) Int. Cl. A61K 31/732; A61K 45/06

(52) U.S. Cl. CPC A61K 31/732; A61K 45/06

(57) ABSTRACT

The invention provides methods for the treatment of diseases and conditions relating to galectin 3 using a galectin-3 inhibitor, such as a modified pectin (e.g., GCS-100). Also described are methods for conjoint therapy, and methods for assessing and/or monitoring the effects of a galectin-3 inhibitor, e.g., to adapt the dosing regimen of the inhibitor during therapy.

### RELATED APPLICATION

This application claims the benefit of U.S. Provisional Application No. 61/950,806, filed Mar. 10, 2014, the contents of which are hereby incorporated by reference.

### BACKGROUND OF THE INVENTION

In developed countries, the continuing rise in individuals diagnosed with hypertension, hyperlipidemia, and diabetes has contributed to an overall increase in the incidence of kidney disorders, such as chronic kidney disease (CKD), NASH, and end-stage renal disease (ESRD). (Tumlin et al., (2013) “Cardiorenal Syndrome Type 4: insights on clinical presentation and pathophysiology from the Eleventh Consensus Conference of the acute dialysis quality initiative,” Contrib Nephrol. 2013; 182:158-73). The rise in the prevalence of chronic kidney disease (CKD) and end-stage renal disease (ESRD) is a global medical and epidemiological problem (Redon et al., (2006) “ERIC-HTA 2003 study investigators: kidney function and cardiovascular disease in the hypertensive population: the ERIC-HTA study,” J. Hypertens 24:663-669). In the United States it is estimated that up to 13% of the population (30 million people) have CKD. A growing body of evidence shows that declining renal function is an independent risk factor for cardiovascular disease. Patients with CKD are at higher risk for death following myocardial infarction and patients experiencing even transient renal dysfunction have increased long-term risk for CVD (Id.; Mathew et al., (2002) “Coronary intervention incidence and prognostic importance of acute renal failure after percutaneous coronary intervention,” (Circulation 105:2259-2264.)

While the mechanisms leading to CKD in patients are not fully known, there is a growing awareness of the role of multiple signaling pathways that act in the renal response to compensate for impaired glomerular filtration rate (GFR) and renal injury. Extensive scientific research has focused on unique pathophysiologic mechanisms of these pathways with the intent to devise new strategies for the treatment the kidney disorders (Ronco, et al., “Cardio-renal syndromes: report from the consensus conference of Acute Dialysis Quality Initiative,” Eur Heart J 31:703-771).

These physiologic responses can lead to activation of multiple compensatory pathways including upregulation of the renin-angiotensin-aldosterone axis (RAAS) and sympathetic nervous system as well as activation of the calcium-parathyroid axis. (Tumlin et al., (2013) “Cardiorenal Syndrome Type 4: insights on clinical presentation and pathophysiology from the Eleventh Consensus Conference of the acute dialysis quality initiative,” Contrib Nephrol. 2013; 182:158-73).

Several recent studies have shown that increases in circulating levels of galectin-3 are associated with worse outcomes in patients with end-stage renal disease (ESRD) (de Boer et al, 2011). Additionally, a number of preclinical studies using multiple animal models of kidney disorder (unilateral ureteral obstruction [UUO], ischemia reperfusion [I/R], and renal transplant) have demonstrated a direct, causal role of galectin-3 expression and secretion by macrophages in the formation of tissue fibrosis leading to kidney failure. Specifically, animals that have been genetically engineered to lack galectin-3 do not exhibit scar formation (fibrosis) after kidney injury or transplantation and, instead, show a reduction in proinflammatory cytokine expression and an improvement in kidney function compared to control mice that express galectin-3 (Henderson et al, 2008, Dang et al, 2012, Fernandes Bertocchi et al, 2008).

These findings collectively underscore the potential of indirectly or directly targeting galectin-3 in the treatment of kidney disorders. Because the ability to downregulate galectin-3 may alleviate renal injury and increase renal function, there is a great need in the art to identify compounds that target galectin-3, or galectin-3 mediated signaling pathways, in order to appropriately determine an efficacious and cost-effective course of therapeutic treatment.

### SUMMARY OF THE INVENTION

The invention described herein provides a safe and effective treatment of kidney disorders using galectin-3 inhibitors, particularly modified pectins, such as GCS-100. The invention further provides combination therapies for treating a kidney disorder with a galectin-3 inhibitor or modified pectin conjointly with one or more additional therapeutic agents useful in the treatment of cancer, cardiovascular disease, infection, inflammation, fibrosis, and renal injury. Compositions and articles of manufacture, including kits, relating to the methods for treating kidney disorder are also contemplated as part of the invention.

In certain embodiments, the galectin-3 inhibitor is administered at a dose that preferentially affects galectin-3 levels and/or activity relative to other galectins, especially galectin-9, e.g., because the agent inhibits galectin-3 levels and/or activity to a greater extent than it inhibits galectin-9 levels and or activity. For example, the IC₅₀ of the agent against galectin-9 may be at least 2, 3, 5, 10, 20, 50, 100, or even over 100 times greater than its IC₅₀ against galectin-3. Without wishing to be bound by theory, inhibiting galectin-9 levels and/or activity may induce undesirable side effects, and so it may be desirable to inhibit galectin-3 levels and/or activity to a therapeutically effective extent without substantially inhibiting galectin-9 levels and/or activity. Accordingly, in some embodiments, the methods described herein include measuring galectin-9 levels in a patient treated with a galectin-3 inhibitor, to determine whether galectin-9 levels and/or activity have been affected to a clinically significant extent. If the measurement shows that galectin-9 levels and/or activity have been significantly affected, one or more subsequent doses of the galectin-3 inhibitor may be reduced relative to the dose administered prior to the measurement.

One aspect of the invention provides a method for treating a kidney disorder in a patient, comprising: administering to the patient at least one galectin-3 inhibitor.

In some embodiments, the kidney disorder is selected from NASH (non-alcoholic steatohepatitis), kidney failure, CKD (chronic kidney disease), hepatorenal syndrome, acidosis, ARF (Acute renal failure), Agenesis, Alport syndrome, Amyloidosis, Analgesic nephropathy, Anti-GBM disease (Goodpasture disease), Anti-phospholipid syndrome, Atheroemboli (Cholesterol emboli), Bartter syndrome, Benign familial haematuria, Berger's disease, Brescia-Cimino fistula, Calciphylaxis, Chronic pyelonephritis (Reflux nephropathy), CRF (Chronic renal failure), Chronic renal insufficiency, Conservative management, Crescentic nephritis (RPGN (Rapidly progressive glomerulonephritis)), Cystitis, Cysts in the kidneys, Dense deposit disease or MCGN (mesangiocapillary glomerulonephritis), Diabetes insipidus, Diabetic nephropathy, Dysuria, Edema, ESRD or ESRF (End Stage Renal Disease or End Stage Renal Failure), Fabry disease, Fanconi syndrome, Fibrillary nephritis, FSGS (Focal Segmental Glomerulosclerosis), Gitelman syndrome, Glomerulonephritis, Haematuria, HUS (Haemolytic uraemic syndrome), Hydronephrosis, Henoch-Schonlein purpura, Hepatorenal syndrome, Hypemephroma, Hypoplasia, IgA nephropathy (Berger's disease), Interstitial nephritis, Loin pain haematuria syndrome, Malignant hypertension, Medullary sponge kidney, Membranous nephropathy, Membranoproliferative glomerulonephritis, MCGN (Mesangiocapillary glomerulonephritis), MPA (Microscopic polyangiitis), Nephropathy, Nephrotic syndrome, Nutcracker syndrome, Oliguria, Osteodystrophy, Page kidney, Polyarteritis, (PKD) Polycystic kidney disorder, Post-infectious glomerulonephritis, Prune belly syndrome, Pyelonephritis, Reflux nephropathy, Renal tubular acidosis, Retroperitoneal fibrosis, Sarcoidosis, Schonlein-Henoch purpura, scleroderma renal crisis, Sjogren's syndrome, Systemic sclerosis, Systemic vasculitis, Thin GBM disease, Thrombotic thrombocytopenic purpura, TTP (Thrombotic Thrombocytopenic Purpura), Tuberous sclerosis, Urethritis, Vasculitis, and Wegener's granulomatosis.

In some embodiments, the patient has CKD.

In some embodiments, the patient has NASH.

In some embodiments, the patient has a baseline eGFR (glomerular filtration rate) of about 15-44 mL/min/1.73 m².

In some embodiments, the galectin-3 inhibitor is a modified pectin.

In some embodiments, the backbone of the modified pectin comprises homogalacturonan and/or rhamnogalacturonan I.

In some embodiments, the modified pectin is de-esterified and partially depolymerized, so as to have a disrupted rhamnogalacturonan backbone.

In some embodiments, the modified pectin has an average molecular weight between 50 and 200 kDa, preferably between 80 and 150 kDa.

In some embodiments, the modified pectin is substantially free of modified pectins having molecular weights below 25 kDa.

In some embodiments, the modified pectin is GCS-100.

In some embodiments, the modified pectin is made by passing modified or unmodified pectin through a tangential flow filter.

In some embodiments, the method comprises administering the modified pectin at a dose of about 0.1 to 2 mg/m².

In some embodiments, the dose is about 1.5 mg/m².

In some embodiments, the dose is about 1-10 mg.

In some embodiments, the dose is 1, 2, 3, 4, 5, 6, 7, 8, 9, or 10 mg, preferably 1, 3, or 9 mg.

In some embodiments, the galectin-3 inhibitor is administered weekly or biweekly.

In some embodiments, the galectin-3 inhibitor is administered weekly for an induction phase and then biweekly for a maintenance phase.

In some embodiments, the induction phase is 1-3 months, preferably 2 months.

In some embodiments, the maintenance phase is at least 1 month, preferably at least 3 months, or even six months or more.

In some embodiments, the at least one galectin-3 inhibitor is administered in an amount that reduces a level of uric acid in serum of the patient.

In some embodiments, the at least one galectin-3 inhibitor is administered in an amount that reduces a level of BUN in serum of the patient.

In some embodiments, the at least one galectin-3 inhibitor is administered in an amount that causes a change in GFR in the patient.

In some embodiments, the at least one galectin-3 inhibitor is administered in an amount that reduces a level of galectin-3 in serum of the patient.

In some embodiments, the at least one galectin-3 inhibitor is administered in an amount that reduces an expression level of galectin 3 in the patient.

In some embodiments, the at least one galectin-3 inhibitor is administered in an amount that reduces an activity of galectin-3 in the patient.

In some embodiments, the concentration, expression level, or activity of galectin-3 is reduced 0.5, 1, 2, 3, 4, or 5-fold relative to control.

In some embodiments, the method further comprises 1) measuring the concentration, level, or activity of galectin-3 before administering the galectin-3 inhibitor and 2) measuring the concentration, level, or activity of galectin-3 after administering the galectin-3 inhibitor.

In some embodiments, a decrease in the concentration, level, or activity of galectin-3 after administering the galectin-3 inhibitor indicates that the dose of galectin-3 inhibitor is an effective dose of galectin-3 inhibitor for the treatment of kidney disorder in a patient.

In some embodiments, an increase in the concentration, level, or activity of galectin-3 after administering the galectin-3 inhibitor indicates that the dose of galectin-3 inhibitor is an ineffective dose of galectin-3 inhibitor for the treatment of kidney disorder in a patient.

In some embodiments, the method further comprises administering to the patient a second dose of the galectin-3 inhibitor in a lower amount than in the prior administration.

In some embodiments, the method further comprises administering an additional therapeutic agent.

In some embodiments, the additional therapeutic agent is useful for the treatment of cardiovascular disease, renal failure, cancer, inflammation, fibrosis, or infection.

In some embodiments, the additional therapeutic agent is selected from an antioxidant, anti-inflammatory drug, chemotherapeutic, anti-infective, antibiotic, or anti-fibrosis drug.

In some embodiments, the method comprises administering the galectin-3 inhibitor concurrently with the therapeutic agent.

In some embodiments, the method comprises administering the galectin-3 inhibitor subsequent to administration of the therapeutic agent.

In some embodiments, the method comprises administering the therapeutic agent subsequent to administration of the galectin-3 inhibitor.

In some embodiments, the method comprises administering multiple doses of the galectin-3 inhibitor over a period of at least 8 weeks.

In some embodiments, the method comprises administering the galectin-3 inhibitor weekly.

In some embodiments, the galectin-3 inhibitor is administered by injection or intravenous infusion.

In some embodiments, the galectin-3 inhibitor is administered by intravenous infusion.

It is contemplated that all embodiments described herein, including those described under different aspects of the invention, can be combined with one another where not specifically prohibited.

### BRIEF DESCRIPTION OF THE DRAWINGS

FIG. 1 depicts the family of known mammalian galectins.

FIG. 2 schematically depicts the structure of GCS-100 unbound and bound to galectin-3.

FIG. 3 shows the GCS-100 concentration versus baseline galectin-3 following a single 1.5 mg/m² dose in cancer patients.

FIG. 4 shows the GCS-100 concentration versus baseline galectin-3 following a single 30 mg/m² dose in cancer patients.

FIG. 5 shows change in eGFR over time.

### DETAILED DESCRIPTION OF THE INVENTION

Provided herein are methods for treating kidney disorders, such as chronic kidney disease or NASH, using galectin-3 inhibitors, particularly modified pectins, such as GCS-100. The invention further provides combination therapies for treating a kidney disorder with a galectin-3 inhibitor or modified pectin conjointly with one or more additional therapeutic agents useful in the treatment of cancer, cardiovascular disease, infection, inflammation, fibrosis, and renal injury. Also described are methods for assessing and/or monitoring the effects of a galectin-3 inhibitor, e.g., to adapt the dosing regimen of the inhibitor during therapy. Compositions and articles of manufacture, including kits, relating to the methods for treating kidney disorder are also contemplated as part of the invention.

Various aspects of the invention are described in further detail herein.

### I. Definitions

Unless otherwise defined herein, scientific and technical terms used in this application shall have the meanings that are commonly understood by those of ordinary skill in the art. Generally, nomenclature and techniques relating to chemistry, molecular biology, cell and cancer biology, immunology, microbiology, pharmacology, and protein and nucleic acid chemistry, described herein, are those well known and commonly used in the art.

Throughout this specification, the word “comprise” or variations such as “comprises” or “comprising” may be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The term “including” is used to mean “including but not limited to.” “Including” and “including but not limited to” are used interchangeably.

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The “baseline” is the last assessment taken prior to the first study drug administration.

The “change from baseline” is the arithmetic difference between a post-baseline value and the baseline value: Change from Baseline=(Post-baseline Value−Baseline Value) Percentage Change from Baseline=[(Post-baseline Value−Baseline Value)/Baseline Value]×100.

The “Body Surface Area,” or BSA is defined by the formula:

$$BSA = \sqrt{\frac{Ht ( cm ) \times Wt ( kg )}{3600}}$$

A “clinical response” as used herein is refers to an indicator of therapeutic effectiveness of an agent. For example, a clinical response may be determined by the change in estimated glomerular filtration rate (eGFR) from baseline relative to control after administration of a modified pectin, such as GCS-100, for 8 weeks in patients with chronic kidney disease (CKD) and baseline eGFR of about 15-44 mL/min/1.73 m². A clinical response may be the safety and tolerability of a modified pectin administered for 8 weeks relative to control in patients with CKD. In certain embodiments, a clinical response is the measurement of the effect of a modified pectin relative to control on 1) circulating galectin-3 levels; 2) serum markers; and/or 3) markers of inflammation, fibrosis, and renal injury.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The terms “conjoint therapy” and “combination therapy,” as used herein, refer to the administration of two or more therapeutic substances, e.g., a galectin-3 inhibitor or modified pectin, and another drug used in the treatment of inflammation, fibrosis, renal injury, or cancer. The other drug(s) may be administered concomitant with, prior to, or following the administration of a galectin-3 inhibitor or modified pectin.

The term “dose,” as used herein, refers to an amount of a therapeutic agent, such as a galectin-3 inhibitor or modified pectin (e.g., GCS-100), which is administered to a subject.

The term “dosing,” as used herein, refers to the administration of a therapeutic agent, such as galectin-3 inhibitor or modified pectin (e.g., GCS-100), to achieve a therapeutic objective (e.g., treatment of a kidney disorder). The level of dosing could be based on the baseline level of galectin-3. One way of determining an appropriate dose would be to measure baseline galectin to determine a target dose, followed by additional measurements after administration to determine the dose's effect on galectin-3.

A “dosing regimen” describes a schedule for administering a therapeutic agent, such as a galectin-3 inhibitor or modified pectin (e.g., GCS-100), e.g., a treatment schedule over a prolonged period of time or throughout the course of treatment, e.g., administering a first dose of a galectin-3 inhibitor or modified pectin (e.g., GCS-100) at week 0 followed by a second dose of a galectin-3 inhibitor or modified pectin (e.g., GCS-100) on a weekly or biweekly dosing regimen.

A “glomerular filtration rate,” or GFR, is a test used to check how well the kidneys are functioning. Specifically, it estimates how much blood passes through the glomeruli each minute. The glomeruli are the tiny filters in the kidneys that filter waste from the blood. GFR may be measured every 2 weeks, 4 weeks, 6 weeks, 8 weeks, 10 weeks, 12 weeks, 16 weeks, 20 weeks, 24 weeks, 26 weeks, 28 weeks, 32 weeks, 34 weeks, 36 weeks, 42 weeks, 44 weeks, 48 weeks, 50 weeks, 52 weeks, 56 weeks, 57 weeks, etc. Preferably, the GFR is measured at 0 weeks, 50 weeks, and 57 weeks.

The term “fixed dose” or “total body dose” refers to a dose which is a constant amount of a therapeutic agent delivered with each administration to the subject being treated. In certain embodiments, a galectin-3 inhibitor or modified pectin (e.g., GCS-100), is administered to the subject at a fixed dose ranging from 0.1 mg/m² to 30 mg/m². In certain embodiments, a modified pectin or galectin-3 inhibitor, is administered to the subject in a fixed dose of 0.1 mg/m², 0.5 mg/m², 1 mg/m², 3 mg/m², 6 mg/m², 9 mg/m², 12 mg/m², 15 mg/m², 18 mg/m², 21 mg/m², 24 mg/m², 27 mg/m², 30 mg/m², 35 mg/m², 40 mg/m², 50 mg/m², 60 mg/m², 70 mg/m², 80 mg/m², 90 mg/m², 100 mg/m², 110 mg/m², 120 mg/m², 130 mg/m², 140 mg/m², 150 mg/m², 160 mg/m², 170 mg/m², 180 mg/m², 190 mg/m², 200 mg/m², etc. Ranges of values between any of the aforementioned recited values are also intended to be included in the scope of the invention, e.g., 0.2 mg/m², 0.6 mg/m², 1.9 mg/m², 4 mg/m², 8 mg/m², 10 mg/m², 13 mg/m², 17 mg/m², 20 mg/m², 23 mg/m², 25 mg/m², 26 mg/m², 28 mg/m², 32 mg/m², 45 mg/m², 55 mg/m², 65 mg/m², 75 mg/m², 85 mg/m², 95 mg/m², 105 mg/m², 115 mg/m², 125 mg/m², 135 mg/m², 145 mg/m², 155 mg/m², 165 mg/m², 175 mg/m², 185 mg/m², 195 mg/m², 205 mg/m², as are ranges based on the forementioned doses, e.g., 0.1-5 mg/m², 5-10 mg/m², 10-15 mg/m², 15-20 mg/m², 20-25 mg/m², 25-30 mg/m², 30-80 mg/m², 80-120 mg/m², 120-150 mg/m², 150-175 mg/m², 175-200 mg/m². The total body dose should not exceed 1 g/m² weekly or 200 mg/m² daily times 5.

The term “induction dose” or “loading dose,” used interchangeably herein, refers to the first dose(s) of a modified pectin or galectin-3 inhibitor (e.g., GCS-100) which is initially used to treat a kidney disorder. The loading dose may, for example, be administered during an induction phase. The loading dose may be larger in comparison to the subsequent maintenance or treatment dose. The induction dose can be a single dose or, alternatively, a set of doses. For example, a 1.5 mg/m² dose may be administered as a single 1.5 mg/m² dose, as two doses of 0.75 mg/m² each, or four doses of 0.375 mg/m² each. In certain embodiments, an induction dose is subsequently followed by administration of smaller doses of a modified pectin or galectin-3 inhibitor (e.g., GCS-100), e.g., the treatment or maintenance dose(s). The induction dose is administered during the induction or loading phase of therapy. The induction phase may be followed by a maintenance phase.

Those “in need of treatment” include mammals, such as humans, already having kidney disorder, including those in which the disease or disorder is to be prevented, e.g., those identified as being at risk of developing the disease or disorder.

As used herein, the term “kidney disorder” refers to any nephropathy, disease, condition, illness, infection, inflammation, deterioration, fibrosis, injury, or scarring of the kidney. Kidney disorder may include, but not limited, to the following NASH (non-alcoholic steatohepatitis), kidney failure, CKD (chronic kidney disease), hepatorenal syndrome, acidosis, ARF (Acute renal failure), Agenesis, Alport syndrome, Amyloidosis, Analgesic nephropathy, Anti-GBM disease (Goodpasture disease), Anti-phospholipid syndrome, Atheroemboli (Cholesterol emboli), Bartter syndrome, Benign familial haematuria, Berger's disease, Brescia-Cimino fistula, Calciphylaxis, Chronic pyelonephritis (Reflux nephropathy), CRF (Chronic renal failure), Chronic renal insufficiency, Crescentic nephritis RPGN (Rapidly progressive glomerulonephritis), Cystitis, Cysts in the kidneys, Dense deposit disease or MCGN (mesangiocapillary glomerulonephritis), Diabetes insipidus, Diabetic nephropathy, Dysuria, Edema, ESRD or ESRF (End Stage Renal Disease or End Stage Renal Failure), Fabry disease, Fanconi syndrome, Fibrillary nephritis, FSGS (Focal Segmental Glomerulosclerosis), Gitelman syndrome, Glomerulonephritis, Haematuria, HUS (Haemolytic uraemic syndrome), Hydronephrosis, Henoch-Schonlein purpura, Hepatorenal syndrome, Hypernephroma, Hypoplasia, IgA nephropathy (Berger's disease), Interstitial nephritis, Loin pain haematuria syndrome, Malignant hypertension, Medullary sponge kidney, Membranous nephropathy, Membranoproliferative glomerulonephritis, MCGN (Mesangiocapillary glomerulonephritis), MPA (Microscopic polyangiitis), Nephropathy, Nephrotic syndrome, Nutcracker syndrome, Oliguria, Osteodystrophy, Page kidney, Polyarteritis, Polycystic kidney disorder (PKD), Post-infectious glomerulonephritis, Prune belly syndrome, Pyelonephritis, Reflux nephropathy, Renal tubular acidosis, Retroperitoneal fibrosis, Sarcoidosis, Schonlein-Henoch purpura, scleroderma renal crisis, Sjogren's syndrome, Systemic sclerosis, Systemic vasculitis, Thin GBM disease, TTP (Thrombotic Thrombocytopenic Purpura), Tuberous sclerosis, Urethritis, Vasculitis, and Wegener's granulomatosis.

The term “lectin” refers to a protein found in the body that specifically interacts with carbohydrate sugars located in, on the surface of, and in between cells. This interaction causes the cells to change behavior, including cell movement, proliferation, and other cellular functions. Interactions between lectins and their target carbohydrate sugars occur via a carbohydrate recognition domain (CRD) within the lectin. Galectins are a subfamily of lectins.

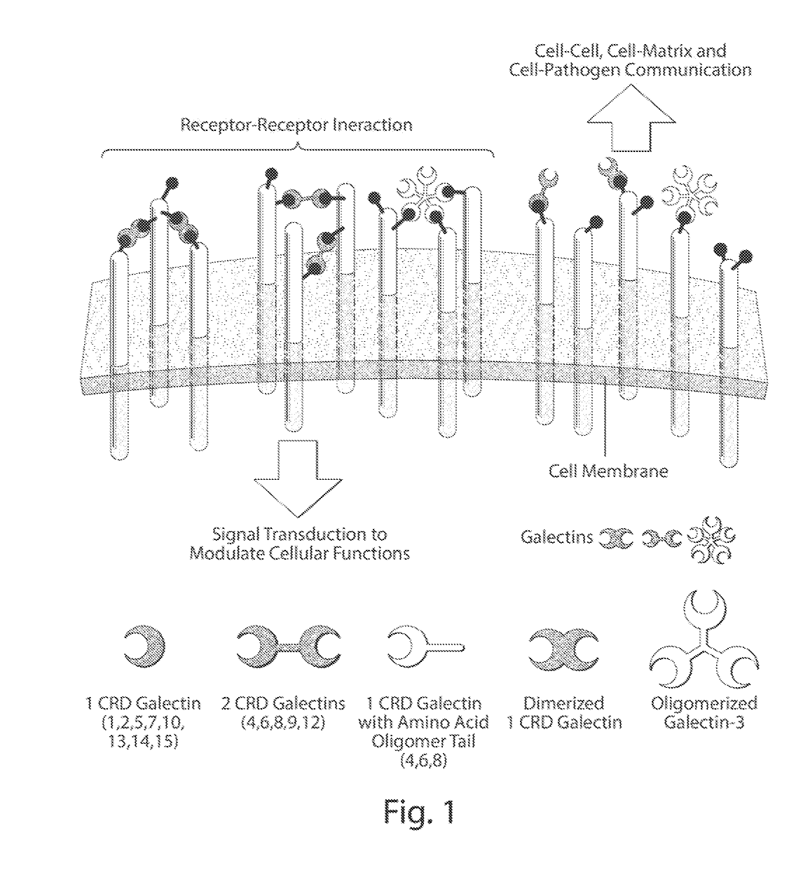

The term “galectins” are a subfamily of lectins that have a CRD that bind specifically to β-galactoside sugar molecules. Galectins have a broad range of functions, including mediation of cell survival and adhesion, promotion of cell-cell interactions, growth of blood vessels, and regulation of the immune system and inflammatory response (Leffler et. al., 2004). Currently, there are 15 known mammalian galectins, which can be divided into three subclasses: those with one CRD (galectins 1, 2, 5, 7, 10, 13, 14, and 15), those with two CRDs (galectins 4, 6, 8, 9, and 12), and those with one CRD and a second domain comprising an amino acid tail (galectin 3), as depicted in FIG. 1. At low concentrations, galectins exist as monomers. However, at higher concentrations, they exist as dimers and oligomers (FIG. 1) and, thus, form lattice-like networks with β-galactoside-containing receptors within a cell and between the cell and its environment (FIG. 1). As such, at low concentrations, galectins may have a different biological function that changes upon upregulation and overexpression (Rabinovich et. al., 2007).

The term “maintenance therapy” or “maintenance dosing regimen” refers to a treatment schedule for a subject or patient diagnosed with a kidney disorder, to enable them to maintain their health in a given state, e.g., reduced renal injury or achieving a clinical response. For example, a maintenance therapy of the invention may enable a patient to maintain their health in a state which is completely or substantially free of symptoms. Alternatively, a maintenance therapy of the invention may enable a patient to maintain his health in a state where there is a significant reduction in symptoms associated with the disease relative to the patient's condition prior to receiving therapy.

The term “maintenance phase” or “treatment phase,” as used herein, refers to a period of treatment comprising administration of a modified pectin or galectin-3 inhibitor (e.g., GCS-100) to a subject in order to maintain a desired therapeutic effect, e.g., improved symptoms associated with kidney disorder. The maintainance phase may be preceded by an induction phase, which is typically a dose larger than a maintenance dose, e.g., with the aim of quickly raising a patient's plasma level of a therapeutic agent, such as a modified pectin, from a baseline level (e.g., 0) into a therapeutically effective window, which is then maintained by administration in the maintenance phase.

The term “maintenance dose” or “treatment dose” is the amount of a modified pectin or galectin-3 inhibitor (e.g., GCS-100) taken by a subject to maintain or continue a desired therapeutic effect. A maintenance dose can be a single dose or, alternatively, a set of doses. A maintenance dose is administered during the treatment or maintenance phase of therapy. Typically, a maintenance dose(s) is smaller than the induction dose(s) and maintenance doses may be equal to each other when administered in succession.

The phrase “multiple-variable dose” includes different doses of a modified pectin or galectin-3 inhibitor (e.g., GCS-100) which are administered to a subject for therapeutic treatment. “Multiple-variable dose regimen” or “multiple-variable dose therapy” describes a treatment schedule which is based on administering different amounts of modified pectin or galectin-3 inhibitor (e.g., GCS-100) at various time points throughout the course of treatment.

The term “pharmaceutically effective amount” or “therapeutically effective amount” refers to an amount of the composition or therapeutic agent, such as a galectin-3 inhibitor, effective to treat kidney disorder in a patient, e.g., improving renal function, and/or effecting a beneficial and/or desirable alteration in the general health of a patient suffering from a kidney disease. A “pharmaceutically effective amount” or “therapeutically effective amount” also refers to an amount that improves the clinical symptoms of a patient.

The phrase “pharmaceutically acceptable excipient” as used herein means a pharmaceutically acceptable material, composition or vehicle, such as a liquid or solid filler, diluent, lubricant, binder, carrier, humectant, disintegrant, solvent or encapsulating material, that one skilled in the art would consider suitable for rendering a pharmaceutical formulation suitable for administration to a subject. Each excipient must be “acceptable” in the sense of being compatible with the other ingredients of the formulation, as well as “pharmaceutically acceptable” as defined above. Examples of materials which can serve as pharmaceutically acceptable excipients include but are not limited to: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; silica, waxes; oils, such as corn oil and sesame oil; glycols, such as propylene glycol and glycerin; polyols, such as sorbitol, mannitol and polyethylene glycol; esters, such as ethyl oleate and ethyl laurate; agar; buffering agents; alginic acid; pyrogen-free water; isotonic saline; Ringer's solution; and other non-toxic compatible substances routinely employed in pharmaceutical formulations.

The term “preventing” is art-recognized, and when used in relation to a medical condition such as a kidney disorder, is well understood in the art, and includes administration of a composition which reduces the frequency of, or delays the onset of, symptoms of a medical condition in a subject relative to a subject which does not receive the composition. Prevention of renal toxicity includes, for example, removing toxic substances from the kidneys to avoid a deleterious effect of those substances on the kidneys and their function.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom). Prophylatic and therapeutic treatment may be used in conjunction with known methods of relieving kidney dysfunction, such as, but not limited to, angioplasty, haemodialysis, haemofiltration, lithotripsy, dialysis, and palliative care.

The terms “subject” and “patient”, as used herein, are used interchangeably. In certain embodiments, a subject refers to an individual who may be treated therapeutically with a modified pectin or galectin-3 inhibitor (e.g., GCS-100).

By “substantially free” of modified pectins having a certain molecular weight below a certain number, it is meant that the composition has less than 1%, preferably less than 0.5% or even less than 0.1%, of modified pectins having a molecular weight below that number.

A “therapeutically effective amount” of a compound, such as a modified pectin of the present invention, with respect to the subject method of treatment, refers to an amount of the compound(s) in a preparation which, when administered as part of a desired dosage regimen to a subject achieves a therapeutic objective (e.g., treatment of a kidney disorder). A therapeutically effective amount may be determined by measuring baseline galectin-3 levels to determine a target dose, followed by additional measurements after administration to determine the effect of the dose on galectin-3. In such embodiments, if the patient's galectin-3 level or activity is decreased, inhibited, or reduced, then the dose is a therapeutically effective amount.

The term “treatment,” as used within the context of the present invention, is meant to include therapeutic treatment, as well as prophylactic or suppressive measures,

### III. Galectin-3 Inhibitors

In certain embodiments of the present invention, the galectin-3 inhibitor is an agent that binds to and inhibits galectin-3, e.g., by reducing its anti-apoptotic activity. Such agents can work, for example, by preventing intracellular signal transduction pathways and/or translocation of galectin-3. Merely to illustrate, the agent can be one which inhibits the multimerization of galectin-3 and/or its interaction of galectin-3 with an anti-apoptotic Bcl-2 protein, such as Bcl-2 or bcl-xL. It may also be an agent that inhibits phosphorylation of galectin-3, such as by inhibiting phosphorylation of galectin-3 at Ser-6. At a gross mechanistic level, the inhibitor can be an agent that inhibits translocation of galectin-3 between the nucleus and cytoplasm or inhibits galectin-3 translocation to the perinuclear membranes and inhibits cytochrome C release from mitochondria. The inhibitor can also be an agent that induces proliferation of fibroblasts, e.g., by binding to and inhibiting galectin-3.

One class of galectin-3 inhibitors contemplated by the present invention is polymers, particularly carbohydrate-containing polymers, that bind to galectin-3 and inhibit its anti-apoptotic activity. Materials useful in the present invention may generally comprise natural or synthetic polymers and oligomers. Preferably, such polymers are very low in toxicity.

A preferred class of polymers for the practice of the present invention is carbohydrate-derived polymers that contain an active galectin-binding sugar site, but that have higher molecular weights than simple sugars, making them capable of sustained blocking, activation, suppression, or other interaction with the galectin protein. A preferred class of therapeutic materials comprises oligomeric or polymeric species of natural or synthetic origin, rich in galactose or arabinose, such as pectin. Such materials may preferably have a molecular weight in the range of up to 500,000 daltons and, more preferably, in the range of up to 100,000 daltons. One particular material comprises a substantially demethoxylated polygalacturonic acid backbone which may be interrupted by rhamnose with galactose-terminated side chains pendent therefrom. Another particular material comprises a homogalacturonan backbone with or without side chains pendent therefrom.

Pectin is a complex carbohydrate having a highly branched structure comprised of a polygalacturonic backbone with numerous branching side chains dependent therefrom. The branching creates regions which are characterized as being “smooth” and “hairy.” It has been found that pectin can be modified by various chemical, enzymatic or physical treatments to break the molecule into smaller portions having a more linearized, substantially demethoxylated, polygalacturonic backbone with pendent side chains of rhamnose residues having decreased branching. The resulting partially depolymerized pectin is known in the art as modified pectin.

In certain embodiments, the invention provides a modified pectin comprising rhamnogalacturonan and/or homogalacturonan backbone with neutral sugar side chains, and having a low degree of neutral sugar branching dependent from the backbone. In certain embodiments, the modified pectin is de-esterified and partially depolymerized, so as to have a disrupted rhamnogalacturonan backbone.

In certain embodiments, the modified pectin includes a copolymer of galacturonic acid and rhamnogalacturonan I in which at least some of the galactose- and arabinose-containing sidechains are still attached. In preferred embodiments, the modified pectin has an average molecular weight of 50-200 kD, preferably 70-200 kD, more preferably 70-150 kD as measured by Gel Permeation Chromatography (GPC) with Multi Angle Laser Light Scattering (MALLS) detection.

In certain embodiments, the modified pectin comprises a homogalacturonan backbone with small amounts of rhamnogalacturonan therein, wherein the backbone has neutral sugar side chains having a low degree of branching dependent from the backbone. In particular embodiments, the galacturonic acid subunits of the homogalacturonan backbone have been partially de-esterified.

In certain embodiments, the invention may be described by either or both of formulas I and II below, and it is to be understood that variants of these general formula may be prepared and utilized in accord with the principles described in U.S. Pat. No. 8,128,966.

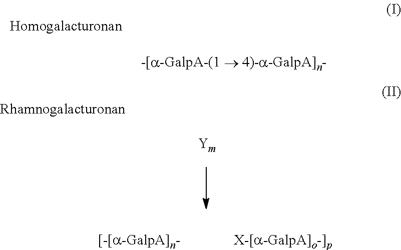

In the formula above, m is ≧0, n, o and p are ≧1, X is α-Rhap; and Yₘ represents a linear or branched chain of sugars (each Y in the chain Yₘ can independently represent a different sugar within the chain). The sugar Y may be, but is not limited to, any of the following: α-Galp, β-Galp, β-Apif, β-Rhap, α-Rhap, α-Fucp, β-GlcpA, α-GalpA, β-GalpA, β-DhapA, Kdop, β-Acef, α-Araf, β-Araf, and α-Xylp.

An exemplary polymer of this type is modified pectin, preferably water-soluble pH-modified citrus pectin. Suitable polymers of this type are disclosed in, for example U.S. Pat. Nos. 5,834,442, 5,895,784, 6,274,566, 6,500,807, 7,491,708, and 8,128,966, U.S. Patent Publication 2002/0107222, and PCT Publications WO 96/01640 and WO 03/000118.

It may be understood that natural pectin does not possess a strictly regular repeating structure, and that additional random variations are likely to be introduced by partial hydrolysis of the pectin, so that the identity of Yₘ and the values of “n” and “o” may vary from one iteration to the next of the p repeating units represented by Formula II above.

Abbreviated sugar monomer names used herein are defined as follows: GalA: galacturonic acid; Rha: rhamnose; Gal: galactose; Api: erythro-apiose; Fuc: fucose; GlcA: glucuronic acid; DhaA: 3-deoxy-D-lyxo-heptulosaric acid; Kdo: 3-deoxy-D-manno-2-octulosonic acid; Ace: aceric acid (3-C-carboxy-5-deoxy-L-lyxose); Ara: arabinose. Italicized p indicates the pyranose form, and italicized f indicates a furanose ring.

U.S. Pat. Nos. 5,895,784, 8,128,966, 8,658,224, 8,409,635, 8,420,133, and 8,187,642, the disclosures of which are incorporated herein by reference, describe modified pectin materials, techniques for their preparation, and use of the material as a treatment for various cancers, and these materials may also be used in the compositions and methods described herein. As described in the '784 patent, modified pectins prepared by a pH-based modification procedure in which the pectin is put into solution and exposed to a series of programmed changes in pH results in the breakdown of the molecule to yield therapeutically effective modified pectin. A preferred starting material is citrus pectin, although it is to be understood that modified pectins may be prepared from pectin obtained from other sources, such as apple pectin. Also, modification may be done by enzymatic treatment of the pectin, or by physical processes such as heating. Further disclosure of modified pectins and techniques for their preparation and use are also found in U.S. Pat. Nos. 5,834,442 and 7,491,708, the disclosures of which are incorporated herein by reference. Modified pectins of this type generally have molecular weights in the range of less than 100 kilodaltons. A group of such materials has an average molecular weight of less than 3 kilodaltons. Another group has an average molecular weight in the range of 1-15 kilodaltons, with a specific group of materials having a molecular weight of about 10 kilodaltons. In certain embodiments, modified pectin has the structure of a pectic acid polymer with some of the pectic side chains still present. In preferred embodiments, the modified pectin is a copolymer of homogalacturonic acid and rhamnogalacturonan I in which some of the galactose- and arabinose-containing sidechains are still attached. The modified pectin may have an average molecular weight of 1 to 500 kilodaltons (kD), preferably 10 to 250 kD, more preferably 50-200 kD or 80-150 kD, and most preferably 80 to 100 kD as measured by Gel Permeation Chromatography (GPC) with Multi Angle Laser Light Scattering (MALLS) detection. In certain embodiments, the modified pectin is a modified apple pectin having an average molecular weight in the range of 20-70 kD. In certain embodiments, the modified pectin may have a average molecular weight in the range of 1-15 kD, while in other embodiments, the modified pectin has an average molecular weight in the range of 15-60 kD. See Gunning, et al., The FASEB Journal, (2009) vol. 23, p. 416, incorporated herein by reference in its entirety, for its discussion of galactans that bind galectin-3. Such galactans may also be used in the compositions and methods described herein.

In certain embodiments, the modified pectin is substantially free of modified pectins having a molecular weight below 25 kDa. The modified pectin may be prepared by passing modified or unmodified pectin through a tangential flow filter.

Degree of esterification is another characteristic of modified pectins. In certain embodiments, the degree of esterification may be between 0 and 80%, between 10 and 60%, between 0 and 50%, or between 20 and 60%, such as 20-45%, or 30-40% esterification.

Saccharide content is another characteristic of modified pectins. In certain embodiments, the modified pectin is composed entirely of a single type of saccharide subunit. In other embodiments, the modified pectin comprises at least two, preferably at least three, and most preferably at least four types of saccharide subunits. For example, the modified pectin may be composed entirely of galacturonic acid subunits. Alternatively, the modified pectin may comprise a combination of galacturonic acid and rhamnose subunits. In yet another example, the modified pectin may comprise a combination of galacturonic acid, rhamnose, and galactose subunits. In yet another example, the modified pectin may comprise a combination of galacturonic acid, rhamnose, and arabinose subunits. In still yet another example, the modified pectin may comprise a combination of galacturonic acid, rhamnose, galactose, and arabinose subunits. In some embodiments, the galacturonic acid content of modified pectin is greater than 50%, preferably greater than 60% and most preferably greater than 80%. In some embodiments, the rhamnose content is less than 25%, preferably less than 15% and most preferably less than 10%; the galactose content is less than 50%, preferably less than 40% and most preferably less than 30%; and the arabinose content is less than 15%, preferably less than 10% and most preferably less than 5%. In certain embodiments, the modified pectin may contain other uronic acids, xylose, ribose, lyxose, glucose, allose, altrose, idose, talose, glucose, mannose, fructose, psicose, sorbose or talalose in addition to the saccharide units mentioned above.

Modified pectin suitable for use in the subject methods may also have any of a variety of linkages or a combination thereof. By linkages it is meant the sites at which the individual sugars in pectin are attached to one another. In some embodiments, the modified pectin comprises only a single type of linkage. In certain preferred embodiments, the modified pectin comprises at least two types of linkages, and most preferably at least 3 types of linkages. For example, the modified pectin may comprise only alpha-1,4 linked galacturonic acid subunits. Alternatively, the modified pectin may comprise alpha-1,4-linked galacturonic acid subunits and alpha-1,2-rhamnose subunits. In another example, the modified pectin may be composed of alpha-1,4-linked galacturonic acid subunits and alpha-1,2-rhamnose subunits linked through the 4 position to arabinose subunits. In another example, the modified pectin may comprise alpha-1,4-linked galacturonic acid subunits and alpha-1,2-rhamnose subunits linked through the 4 position to arabinose subunits with additional 3-linked arabinose subunits. In another example, the modified pectin may comprise alpha-1,4-linked galacturonic acid subunits and alpha-1,2-rhamnose subunits linked through the 4 position to arabinose subunits with additional 5-linked arabinose units. In another example, the modified pectin may comprise alpha-1,4-linked galacturonic acid subunits and alpha-1,2-rhamnose subunits linked through the 4 position to arabinose subunits with additional 3-linked and 5-linked arabinose subunits. In another example, the modified pectin may comprise alpha-1,4-linked galacturonic acid subunits and alpha-1,2-rhamnose subunits linked through the 4 position to arabinose subunits with additional 3-linked and 5-linked arabinose subunits with 3,5-linked arabinose branch points. In another example, the modified pectin may comprise alpha-1,4-linked galacturonic acid subunits and alpha-1,2-rhamnose subunits linked through the 4 position to galactose subunits. In another example, the modified pectin may comprise alpha-1,4-linked galacturonic acid subunits and alpha-1,2-rhamnose subunits linked through the 4 position to galactose subunits with additional 3-linked galactose subunits. In another example, the modified pectin may comprise alpha-1,4-linked galacturonic acid subunits and alpha-1,2-rhamnose subunits linked through the 4 position to galactose subunits with additional 4-linked galactose subunits. In another example, the modified pectin may comprise alpha-1,4-linked galacturonic acid subunits and alpha-1,2-rhamnose subunits linked through the 4 position to galactose subunits with additional 3-linked galactose subunits with 3,6-linked branch points. In another example, the modified pectin may comprise alpha-1,4-linked galacturonic acid subunits and alpha-1,2-rhamnose subunits linked through the 4 position to galactose subunits with additional 4-linked galactose subunits with 4,6-linked branch points. In certain embodiments, the side chains of the modified pectin may comprise uronic acids, galacaturonic acid, glucuronic acid, rhamnose, xylose, ribose, lyxose, glucose, allose, altrose, idose, talose, glucose, mannose, fructose, psicose, sorbose or talalose in addition to the saccharide units described above.

Modified pectins suitable for the compositions and methods described herein may have one or more of the characteristics described above.

Other carbohydrate materials including galactose residues capable of binding and inhibiting galectin-3 can also be employed in the compositions and methods disclosed herein. For example, mannan, dextrans, polygalacturonate, polyglucosamine and other water-soluble polysaccharides (see, for example, U.S. Patent Publication 2005/0043272 to Platt, et al., incorporated herein by reference for the compositions disclosed therein) can be used as galectin-3 inhibitors. The inclusion of target specific carbohydrates, such as, galactose, rhamnose, mannose, or arabinose can be varied to target specific lectin-type receptors on tumor cells, e.g., to modulate relative inhibition of galectin-3 vs. galectin-9. One of skill in the art will recognize that there could be a heterogenous population of carbohydrate residues on the polymer, as is true of some naturally occurring polymers, such as modified pectin and some galactans. Particular polysaccharides include galactomannans (e.g., from Cyamopsis tetragonolobus), arabinogalactan (e.g., from Larix occidentalis), rhamnogalacturonan (e.g., from potato), carrageenan (e.g., from Eucheuma seaweed), and the locust bean gum (e.g., from Ceratonia siliqua).

Alkyl-modified polysaccharides can originate from natural sources and/or be synthetically prepared from naturally occurring carbohydrate polymers. Microbial sources for alkylated polysaccharides are well known to those in the art, see, e.g., U.S. Pat. No. 5,997,881, the teachings of which are incorporated herein in their entirety by reference. Some of the microbial sources have been used in oil spill remediation operations (see Gutnick and Bach “Engineering bacterial biopolymers for the biosorption of heavy metals, Applied Microbiology and Biotechnology, 54 (4) pp 451-460, (2000); also see U.S. Pat. No. 4,395,354, Gutnick, et al. 1983, the entire teachings of which are hereby incorporated herein by reference). These microbes involved in oil spill remediation activities have been referred to as “Emulsans”, in which some of their polysaccharides are O-acylated. Similar alkylated carbohydrates were also isolated from yeast fermentation and are known as sophorolipids.

Another example of suitable polysaccharides is a polysaccharide chain consisting essentially of 2-amino-2,6-dideoxyaldohexose sugar, glucosamine and one or more non-aminated sugars, wherein the amine groups of the aminated sugars are substantially all in acetylated form. The polysaccharide chain is linked with an ester bond to an alkyl moiety consisting of saturated and/or unsaturated chain of about 10 to about 18 carbon atoms of which 50-95% comprises dodecanoic acid and 3-hydroxy-dodecanoic acid. In one particular aspect, the dodecanoic acid is present in an amount greater than the 3-hydroxy-dodecanoic acid.

Optionally, the alkylated polysaccharide can comprise anionic groups, such as phosphate, sulfate, nitrate, carboxyl groups, and/or sulfate groups, while maintaining the hydrophobic moieties.

For example, a synthetic polysaccharide can be esterified with straight or branched alkyl groups of about 8 to about 40 carbon atoms. These alkyl groups may be aliphatic or unsaturated, and optionally may contain one or more aromatic groups. In certain embodiments, the surface of the alkylated polysaccharides can be further derivatized using carbohydrate ligands, e.g., galactose, rhamnose, mannose or arabinose, to further enhance recognition sites by lectins. The polysaccharides of the present invention can be derivatized using alkyl, aryl or other chemical moieties.

In particular embodiments, the polysaccharide can be a galactomannan, as described in U.S. Patent Publications 2003/0064957, 2005/0053664, 2011/0077217, and 2013/0302471, all of which are hereby incorporated by reference herein for the compositions disclosed therein. For example, the molecular weight of the galactomannan can have an average molecular weight in the range of 20-600 kD, for example the galactomannan has a molecular weight in the range of 90 to 415 kD or 40-200 kD, such as an average molecular weight of 83 kD or 215 kD. Suitable galactomannans may be isolated from Gleditsia triacanthos, Ceratonia siliqua, Xanthomonas campestris, Trigonella foenum-graecum, Medicago falcate, or Cyamopsis tetragonoloba or may be prepared from galactomannans isolated therefrom.

In certain such embodiments, the galactomannan may be β-1→4-D-galactomannan and include a ratio of galactose to mannose where mannose is in the range of 1.0-3.0 and galactose is in the range of 0.5-1.5. Alternatively, the galactomannan may have a ratio of 2.6 mannose to 1.5 galactose.

In certain embodiments, the galactomannan has a ratio of 2.2 mannose to 0.9 galactose. Alternatively, the galactomannan may have a ratio of 1.13 mannose to 1 galactose. Alternatively, the galactomannan may have a ratio of 2.2 mannose to 1 galactose.

In certain embodiments, the polysaccharide can be β-1,4-D-galactomannan and include a ratio of mannose to galactose of about 1.7. In certain embodiments, the molecular weight of the galactomannan polysaccharide is in the range of about 4 to about 200 kD. In certain particular embodiments, the galactomannan has an average weight of about 40 to 60 kD. In another aspect, the structure of the galactomannan is a poly-β-1,4 mannan backbone, with side substituents affixed via α-1-6-glycoside linkages. In certain embodiments, the galactomannan polysaccharide can be β-1,4-D-galactomannan. In certain particular embodiments, the polysaccharide is (((1,4)-linked β-D-mannopyranose)17-((1,6)-linked-β-D-galactopyranose)10)12).

Suitable polysaccharides can have side branches of target specific carbohydrates, such as galactose, rhamnose, mannose, or arabinose, to impart recognition capabilities in targeting specific lectin-type receptors on the surface of cells, e.g., to modulate relative inhibition of galectin-3 vs. galectin-9. Branches can be a single unit or two or more units of oligosaccharide.

Yet another suitable polysaccharide is disclosed in U.S. Patent Publication 2005/0282773, hereby incorporated by reference herein for the compositions disclosed therein. Such polysaccharides maybe have a uronic acid saccharide backbone or uronic ester saccharide backbones having neutral monosaccharides connected to the backbone about every one-in-twenty to every one-in-twenty-five backbone units. The resulting polysaccharides may have at least one side chain comprising mostly neutral saccharides and saccharide derivatives connected to the backbone via the about one-in-seven to twenty-five neutral monosaccharides. Some preferred polysaccharides may have at least one side chain of saccharides further having substantially no secondary saccharide branches, with a terminal saccharide comprising galactose, glucose, arabinose, or derivatives thereof. Other preferred polysaccharides may have at least one side chain of saccharides terminating with a saccharide modified by a feruloyl group.

Suitable polysaccharides may have an average molecular weight range of between about 40,000-400,000 dalton with multiple branches of saccharides, for example, branches comprised of glucose, arabinose, galactose, etc., and these branches may be connected to the backbone via neutral monosaccharides such as rhamnose. These molecules may further include a uronic acid saccharide backbone that may be esterified from as little as about 10% to as much as about 90% of uronic acid residues. The multiple branches themselves may have multiple branches of saccharides, the multiple branches optionally including neutral saccharides and neutral saccharide derivatives.

Such polysaccharides may be prepared by a chemical modification procedure that involves a pH-dependent depolymerization into smaller, de-branched polysaccharide molecules, using sequentially controlled pH, temperature and time, e.g., pH 10.0 at 37° C. for 30 minutes and than pH of about 3.5 at 25° C. for 12 hours (see Example 1). An optional alternative modification procedure is hydrolysis of the polysaccharide in an alkaline solution in the presence of a reducing agent such as a potassium borohydride to form fragments of a size corresponding to a repeating subunit (see, e.g., U.S. Pat. No. 5,554,386). The molecular weight range for the chemically modified polysaccharides is in the range of 5 to 60 kD, more specifically, in the range of about 15-40 kD, and more specifically, for example, about 20 kD.

Still other suitable polysaccharides are disclosed in U.S. Patent Publication 2008/0107622, hereby incorporated by reference herein for the compositions disclosed therein. One type of such polysaccharides include galacto-rhamnogalacturonate (GR), a branched heteropolymer of alternating 1,2-linked rhamnose and 1,4-linked Gala residues that carries neutral side-chains of predominantly 1,4-β-D-galactose and/or 1,5-α-L-arabinose residues attached to the rhamnose residues of the RGI backbone. GR side-chains may be decorated with arabinosyl residues (arabinogalactan I) or other sugars, including fucose, xylose, and mannose. These are also referred to in commercial use as pectic material.

Preparation of these polysaccharides may include modifying naturally occurring polymers to reduce the molecular weight for the desired range, adjusting the alkylated groups (demethoxylation or deacetylation), and adjusting side chain oligosaccharides for optimum efficacy. For example, natural polysaccharides may have a molecular weight range of between about 40,000-1,000,000 with multiple branches of saccharides, for example, branches comprised of 1 to 20 monosaccharides of glucose, arabinose, galactose, etc., and these branches may be connected to the backbone via neutral monosaccharides such as rhamnose. These molecules may further include a uronic acid saccharide backbone that may be esterified from as little as about 2% to as much as about 30%. The multiple branches themselves may have multiple branches of saccharides, the multiple branches optionally including neutral saccharides and neutral saccharide derivatives creating mainly hydrophobic entities.

In certain embodiments, a rhamnogalacturonate has a molecular weight range of 2.0 to 200 kD. In specific examples, the rhamnogalacturonate may have an average size molecular weight of about 34 kD or about 135 kD and is obtained through chemical, enzymatic, and/or physical treatments. Starting materials may be obtained via isolation and/or purification from pectic substance of citrus peels, apple pomace, soybean bull, or sugar beets, or other suitable materials, as will be apparent to the skilled artisan.

In certain embodiments, soluble chemically altered galacto-rhamnogalacturonates are prepared by modifying naturally occurring polymers to reduce the molecular weight for the desired range, reducing the alkylated group (de-methoxylation or de acetylation). Prior to chemical modification, the natural polysaccharides may have a molecular weight range of between about 40,000-1,000,000 with multiple branches of saccharides, for example, branches comprised of 1 to 20 monosaccharides of glucose, arabinose, galactose, etc., and these branches may be connected to the backbone via neutral monosaccharides, such as rhamnose. These molecules may further include a single or chain of uronic acid saccharide backbone that may be esterified from as little as about 2% to as much as about 30%. The multiple branches themselves may have multiple branches of saccharides, the multiple branches optionally including neutral saccharides and neutral saccharide derivatives creating mainly hydrophobic entities.

Smaller saccharides can also be used. Suitable compounds include N-acetyllactosamine and its derivatives (see, for example, Sorme, et al., Chembiochem. 2002 Mar. 1; 3(2-3): 183-9, incorporated by reference herein in its entirety, which discloses a range of 3′-amino-N-acetyllactosamine derivatives), as well as oligomeric and polymeric derivatives thereof, such as poly-N-acetyllactosamine.

Other classes of galectin-3 inhibitors that bind to galectin-3 include antibodies specific to galectin-3, peptides and polypeptides that bind to and interfere with galectin-3 activity, and small (preferably less than 2500 amu) organic molecules that bind to and inhibit galectin-3.

To further illustrate, in certain embodiments of the present invention, the subject methods can be carried out using an antibody or fragment thereof that is immunoreactive with galectin-3 and inhibitory for its anti-apoptotic activity.

An exemplary protein therapeutic is described in PCT publication WO 02/100343. That reference discloses certain N-Terminally truncated galectin-3 proteins that inhibit the binding of intact galectin-3 to carbohydrate ligands and thereby also inhibit the multimerization and cross-linking activities of galectin-3 that may be required for its anti-apoptotic activity.

Exemplary small molecule inhibitors of galectin-3 include thiodigalactoside (such as described in Leffler et al., 1986, J. Biol. Chem. 261:10119) and agents described in PCT publication WO 02/057284, incorporated herein by reference for the inhibitors disclosed therein.

In certain preferred embodiments of galectin-3 inhibitors that bind to galectin-3, the inhibitor is selected to having a dissociation constant (Kd) for binding galectin-3 of 10⁻⁶ M or less, and even more preferably less than 10⁻⁷ M, 10⁻⁸ M or even 10⁻⁹ M.

Certain of the galectin-3 inhibitors useful in the present invention act by binding to galectin-3 and disrupting galectin-3's interactions with one or more anti-apoptotic Bcl-2 proteins. A galectin-3 inhibitor may bind directly to the Bcl-2 binding site thereby competitively inhibits Bcl-2 binding. However, galectin-3 inhibitors which bind to the Bcl-2 protein are also contemplated, and include galectin-3 inhibitors that bind to a Bcl-2 protein and either competitively or allosterically inhibit interaction with galectin-3.

As mentioned above, certain of the subject galectin-3 inhibitors exert their effect by inhibiting phosphorylation of galectin-3. The binding of a galectin-3 inhibitor may block the access of kinases responsible for galectin-3 phosphorylation, or, alternatively, may cause conformational change of galectin, concealing or exposing the phosphorylation sites. However, the present invention also contemplates the use of kinase inhibitors which act directly on the kinase(s) that is responsible for phosphorylating galectin-3.

In still other embodiments, inhibition of galectin-3 activity is also achieved by inhibiting expression of galectin-3 protein. Such inhibition is achieved using an antisense or RNAi construct having a sequence corresponding to a portion of the mRNA sequence transcribed from the galectin-3 gene.

In certain embodiments, the galectin-3 inhibitors can be nucleic acids. In certain embodiments, the invention relates to the use of antisense nucleic acid that hybridizes to the galectin-3 mRNA and decreases expression of galectin-3. Such an antisense nucleic acid can be delivered, for example, as an expression plasmid which, when transcribed in the cell, produces RNA which is complementary to at least a unique portion of the cellular mRNA which encodes galectin-3. Alternatively, the construct is an oligonucleotide which is generated ex vivo and which, when introduced into the cell causes inhibition of expression by hybridizing with the mRNA and/or genomic sequences encoding galectin-3. Such oligonucleotide are optionally modified oligonucleotide which are resistant to endogenous nucleases, e.g., exonucleases and/or endonucleases, and is therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., (1988) Biotechniques 6:958-976; and Stein et al., 1988, Cancer Res. 48:2659-2668.

In other embodiments, the invention relates to the use of RNA interference (RNAi) to effect knockdown of expression of the galectin-3 gene. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. RNAi provides a useful method of inhibiting gene expression in vitro or in vivo. As used herein, the term “RNAi construct” is a generic term including small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

RNAi constructs can comprise either long stretches of dsRNA identical or substantially identical to the target nucleic acid sequence or short stretches of dsRNA identical to substantially identical to only a region of the target nucleic acid sequence.

Optionally, the RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 base pairs, or 1 in 10 base pairs, or 1 in 20 base pairs, or 1 in 50 base pairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

The subject RNAi constructs can be “small interfering RNAs” or “siRNAs.” These nucleic acids are around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In some embodiments, the 21-23 nucleotides siRNA molecules comprise a 3′ hydroxyl group. In certain embodiments, the siRNA constructs can be generated by processing of longer double-stranded RNAs, for example, in the presence of the enzyme dicer. For example, the Drosophila in vitro system may be used. In this system, dsRNA is combined with a soluble extract derived from Drosophila embryo, thereby producing a combination. The combination is maintained under conditions in which the dsRNA is processed to RNA molecules of about 21 to about 23 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Methods of chemically modifying RNA molecules can be adapted for modifying RNAi constructs (see, e.g., Heidenreich et al., 1997, Nucleic Acids Res., 25:776-780; Wilson et al., 1994, J. Mol. Recog. 7:89-98; Chen et al., 1995, Nucleic Acids Res. 23:2661-2668; Hirschbein et al., 1997, Antisense Nucleic Acid Drug Dev. 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

In some cases, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In some embodiments, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

The RNAi construct can also be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

Alternatively, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev., 2002, 16:948-58; McCaffrey et al., Nature, 2002, 418:38-9; McManus et al., RNA, 2002, 8:842-50; Yu et al., Proc. Nat'l Acad. Sci. USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In other embodiments, the invention relates to the use of ribozyme molecules designed to catalytically cleave galectin-3 mRNA transcripts to prevent translation of mRNA (see, e.g., PCT International Publication WO90/11364, published Oct. 4, 1990; Sarver et al., 1990, Science 247:1222-1225; and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591. The ribozymes of the present invention also include RNA endoribonucleases (“Cech-type ribozymes”) such as the one which occurs naturally in Tetrahymena thermophila (known as the IVS or L-19 IVS RNA) and which has been extensively described (see, e.g., Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216).

In further embodiments, the invention relates to the use of DNA enzymes to inhibit expression of the galectin-3 gene. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid. Briefly, to design an ideal DNA enzyme that specifically recognizes and cleaves a target nucleic acid, one of skill in the art must first identify the unique target sequence. Preferably, the unique or substantially sequence is a G/C rich of approximately 18 to 22 nucleotides. High G/C content helps insure a stronger interaction between the DNA enzyme and the target sequence. When synthesizing the DNA enzyme, the specific antisense recognition sequence that may target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462.

Other inhibitors may include monoclonal, polyclonal, humanized, and/or chimeric antibodies that bind to galectin-3. The term “antibody,” as used herein, is intended to refer to immunoglobulin molecules comprising four polypeptide chains, two heavy (H) chains and two light (L) chains inter-connected by disulfide bonds. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Representative antibodies are described in further detail in U.S. Pat. Nos. 6,090,382; 6,258,562; and 6,509,015.

The term “antigen-binding portion” or “antigen-binding fragment” of an antibody (or simply “antibody portion”), as used herein, refers to one or more fragments of an antibody that retain the ability to specifically bind to an antigen (e.g., galectin-3). It has been shown that the antigen-binding function of an antibody can be performed by fragments of a full-length antibody. Binding fragments include Fab, Fab′, F(ab′)₂, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CHI domains; (iv) a Fv fragment consisting of the VL and VII domains of a single arm of an antibody, (v) a dAb fragment (Ward et al. (1989) Nature 341:544-546), which consists of a VH domain: and (vi) an isolated complementarity determining region (CDR).

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2: 1121-1123). The antibody portions of the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31: 1047-1058). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In certain embodiments, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In certain embodiments, chimeric antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immmunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific,” “bispecific,” “trispecific,” “tetraspecific,” etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), monoclonal antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

### IV. Serum Markers and Biomarkers

Serum markers may be measured in conjunction with galectin-3 to measure the effect of treatment with a galectin-3 inhibitor, such as a modified pectin (e.g., GCS-100). Whole blood samples may be drawn for determination of the levels of circulating galectin-3, creatinine, BUN, plasma mitogen, and/or other serum markers. Assays for galectin-3 concentration and serum markers may be performed according to the methods described herein and known in the art.

In certain embodiments, the present methods increase the GFR levels by 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 4, 6, 8, or even 10-fold in patients given a low dose of galectin-3 inhibitor, e.g., relative to GFR measured in an untreated patient or a patient treated with placebo.

In certain embodiments, the present methods reduce BUN levels by 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 4, 6, 8, or even 10-fold in patients given a low dose of galectin-3 inhibitor (e.g., 1.5 mg/m² of modified pectin, such as GCS-100), e.g., relative to BUN levels measured in an untreated patient or a patient treated with placebo.

In certain embodiments, the present methods decrease the uric acid levels by 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 4, 6, 8, or even 10-fold in patients given a low dose of galectin-3 inhibitor (e.g., 1.5 mg/m² of modified pectin, such as GCS-100), e.g., relative to uric acid measured in an untreated patient or a patient treated with placebo.

In certain embodiments, the present methods reduce the galectin-3 levels by 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 4, 6, 8, or even 10-fold in patients given a low dose of galectin-3 inhibitor (e.g., 1.5 mg/m² of modified pectin, such as GCS-100), e.g., relative to galectin-3 measured in an untreated patient or a patient treated with placebo.

In certain embodiments, the present methods reduce the urea concentration in serum. In particular, the concentration of urea in serum measured after administration of galectin-3 inhibitor may be reduced by at least 20% relative to urea measured in an untreated patient or a patient treated with placebo.

In certain embodiments, the present methods reduce the absolute and or relative serum creatinine levels. In particular, the relative concentration of creatinine in serum measured after administration of galectin-3 inhibitor may be reduced by at least 5%, at least 10%, at least 20%, at least 30%, at least 40%, or at least 50% relative to creatinine measured in an untreated patient or a patient treated with placebo. In other embodiments, the absolute concentration of creatinine may be reduced by about 0.1-1.0 mg/dl, such as about 0.1-0.5 mg/dl or reduced by more than 0.1 mg/dl, more than 0.2 mg/dl, or more than 0.3 mg/dl.

In certain embodiments, the present methods alter the urinary excretion of proximal tubular injury markers, such as β-2 microglobulin, N-acetyl-β-D-glucoaminidase, and α₁-acid glycoprotein. In particular, the concentration of one or more of the tubular injury markers in urine measured after administration galectin-3 inhibitor may be reduced by at least 20% relative to the tubular injury markers measured in urine after treatment relative to the tubular injury markers measured in an untreated patient or a patient treated with placebo. Other markers may include N-gal, cystatin C, and/or additional urine markers that correlate with kidney activity and/or damage.

### Biomarkers of Inflammation, Fibrosis, and Renal Injury

Determining the presence or level of galectin-3 may also be combined with the detection of one or more other biomarkers for which increased or decreased expression correlates with kidney disorder. The selected biomarker can be a general therapeutic, diagnostic or prognostic marker useful for multiple types of kidney disorder, inflammation, fibrosis, and renal injury. These markers may include, but not be limited to, neutrophil gelatinase-associated lipocalin (NGAL), collagen, interleukin-6 (IL-6), monocyte chemoattractant protein-1 (MCP-1), interferon-γ (IFN-γ), tumor necrosis factor-α (TNF-α), intracellular adhesion molecule-1 (ICAM-1), hemoglobin A1c (HbA1c) and E-selectin.

Those skilled in the art may be able to select one or more useful therapeutic, diagnostic or prognostic markers for measurement in combination with galectin-3. Similarly, three or more, four or more or five or more or a multitude of biomarkers can be used together for determining a diagnosis or prognosis of a patient.

In certain embodiments, the present methods reduce or increase the levels of the biomarkers by 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 4, 6, 8, or even 10-fold in patients given a low dose of galectin-3 inhibitor (e.g., 1.5 mg/m² of modified pectin, such as GCS-100) relative to the levels of the same biomarkers measured in patients administered with placebo.

### V. Galectin-3 and Biomarker Protein Detection Techniques

Methods for the detection of protein, e.g., galectin-3 protein and biomarkers, are well known to those skilled in the art, and include ELISA (enzyme linked immunosorbent assay), RIA (radioimmunoassay), Western blotting, and immunohistochemistry. Immunoassays such as ELISA or RIA, which can be extremely rapid, are more generally preferred. These methods use antibodies, or antibody equivalents, to detect galectin-3 protein. Antibody arrays or protein chips can also be employed, see for example U.S. Patent Application Nos: 20030013208A1; 20020155493A1, 20030017515 and U.S. Pat. Nos. 6,329,209; 6,365,418, herein incorporated by reference in their entirety.

ELISA and RIA procedures may be conducted such that a galectin-3 standard is labeled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, galectin-3 in the sample is allowed to react with the corresponding immobilized antibody, radioisotope- or enzyme-labeled anti-galectin-3 antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In certain embodiments, a method for measuring galectin-3 levels comprises: contacting a biological specimen with an antibody or variant (e.g., fragment) thereof which selectively binds galectin-3, and detecting whether said antibody or variant thereof is bound to said sample and thereby measuring the levels of galectin-3. A method may further comprise contacting the specimen with a second antibody, e.g., a labeled antibody. The method may further comprise one or more steps of washing, e.g., to remove one or more reagents.

Enzymatic and radiolabeling of galectin-3 and/or the antibodies may be effected by any suitable means. Such means may generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and may only yield a proportion of active enzyme.

It may be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect galectin-3 according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Anti-galectin-3 antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of human galectin-3, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy. The results may be quantitated, e.g., as described in the Examples.

Immunohistochemical analysis optionally coupled with quantification of the signal may be conducted as follows. Galectin-3 and biomarker expression may be directly evaluated in the tissue by preparing immunohistochemically stained slides with, e.g., an avidin-biotinylated peroxidase complex system.

Evaluation of the presence of stains, i.e., galectin-3 or biomarker, may also be done by quantitative immunohistochemical investigation, e.g., with a computerized image analyzer (e.g., Automated Cellular Imaging System, ACIS, ChromaVision Medical System Inc., San Juan Capistrano, Calif.) may be used for evaluation of the levels of galectin-3 or biomarker expression in the immunostained tissue samples. Using ACIS, “cytoplasmic staining” may be chosen as program for galectin-3 or biomarker detection. Different areas of immunostained tumor samples may be analyzed with the ACIS system. An average of the ACIS values that is more or less than 1, e.g., about 1.1, 1.2, 1.3, 1.4, 1.5, 2, 2.5, 3, 5, 10, 30, 100 or more indicates an elevated or decreased galectin-3 or biomarker expression.

Other machine or autoimaging systems may also be used to measure immunostaining results for galectin-3. As used herein, “quantitative” immunohistochemistry refers to an automated method of scanning and scoring samples that have undergone immunohistochemistry, to identify and quantitate the presence of a specified biomarker, such as an antigen or other protein. The score given to the sample is a numerical representation of the intensity of the immunohistochemical staining of the sample, and represents the amount of target biomarker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning and analysis systems suitable for use with immunohistochemistry are available in the art. Such systems may include automated staining (see, e.g, the Benchmark™ system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Another method that may be used for detecting and quantitating galectin-3 or biomarker protein levels is Western blotting, e.g., as described in the Examples. Tumor tissues may be frozen and homogenized in lysis buffer. Immunodetection can be performed with a galectin-3 antibody using the enhanced chemiluminescence system (e.g., from PerkinElmer Life Sciences, Boston, Mass.). The membrane may then be stripped and re-blotted with a control antibody, e.g., anti-actin (A-2066) polyclonal antibody from Sigma (St. Louis, Mo.). The intensity of the signal may be quantified by densitometry software (e.g., NIH Image 1.61). After quantification of the galectin-3, biomarker, and control signals (e.g., actin), the relative expression levels of galectin-3 or biomarker are normalized by amount of the actin in each lane, i.e., the value of the galectin-3 or biomarker signal is divided by the value of the control signal. Galectin-3 or biomarker protein expression is considered to be elevated when the relative level is more than 1, e.g., about 1.1, 1.2, 1.3, 1.4, 1.5, 2, 2.5, 3, 5, 10, 30, or even 100. Conversely, galectin-3 or biomarker protein expression is considered to be reduced when the relative level is less than 1, e.g., about 1.1, 1.2, 1.3, 1.4, 1.5, 2, 2.5, 3, 5, 10, 30, or even 100.

Anti-galectin-3 or biomarker antibodies may also be used for imaging purposes, for example, to detect the presence of galectin-3 or biomarkers in cells and tissues of a subject. Suitable labels include radioisotopes, iodine (¹²⁵I, ¹²¹I), carbon (¹⁴C), sulphur (³⁵S), tritium (³H), indium (¹¹²In), and technetium (⁹⁹mTc), fluorescent labels, such as fluorescein and rhodamine, and biotin. Immunoenzymatic interactions can be visualized using different enzymes such as peroxidase, alkaline phosphatase, or different chromogens such as DAB, AEC or Fast Red.

For in vivo imaging purposes, antibodies are not intrinsically detectable from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the patient, such as barium or caesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, may determine the quantity of imaging moiety needed to produce diagnostic images. In the case of a radioisotope moiety, for a human subject, the quantity of radioactivity injected may normally range from about 5 to 20 millicuries of technetium-99m. The labeled antibody or antibody fragment may then preferentially accumulate at the location of cells which contain galectin-3. The labeled antibody or variant thereof, e.g., antibody fragment, can then be detected using known techniques.

Antibodies that may be used to detect galectin-3 include any antibody, whether natural or synthetic, full length or a fragment thereof, monoclonal or polyclonal, that binds sufficiently strongly and specifically to the galectin-3 to be detected, e.g., human galectin-3. An antibody may have a Kd of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to galectin-3 relative to other proteins, such as related proteins, e.g., galectin 1-15.

Antibodies and derivatives thereof that may be used encompasses polyclonal or monoclonal antibodies, chimeric, human, humanized, primatized (CDR-grafted), veneered or single-chain antibodies, phase produced antibodies (e.g., from phage display libraries), as well as functional, i.e., galectin-3 binding fragments, of antibodies. For example, antibody fragments capable of binding to galectin-3 or portions thereof, including, but not limited to Fv, Fab, Fab′ and F(ab′)₂ fragments can be used. Such fragments can be produced by enzymatic cleavage or by recombinant techniques. For example, papain or pepsin cleavage can generate Fab or F(ab′2 fragments, respectively. Other proteases with the requisite substrate specificity can also be used to generate Fab or F(ab′)₂ fragments. Antibodies can also be produced in a variety of truncated forms using antibody genes in which one or more stop codons have been introduced upstream of the natural stop site. For example, a chimeric gene encoding a F(ab′)₂ heavy chain portion can be designed to include DNA sequences encoding the CH, domain and hinge region of the heavy chain.

In some embodiments, agents that specifically bind to galectin-3 or other than antibodies are used, such as peptides. Peptides that specifically bind to galectin-3 can be identified by any means known in the art. For example, specific peptide binders of galectin-3 can be screened for using peptide phage display libraries.

Generally, a reagent that is capable of detecting a galectin-3 or biomarker polypeptide, such that the presence of galectin-3 or other biomarker is detected and/or quantitated, may be used. As defined herein, a “reagent” refers to a substance that is capable of identifying or detecting galectin-3 in a biological sample (e.g., identifies or detects galectin-3 or biomarker mRNA, DNA, and protein). In some embodiments, the reagent is a labeled or labelable antibody which specifically binds to galectin-3 or biomarker polypeptide. As used herein, the phrase “labeled or labelable” refers to the attaching or including of a label (e.g., a marker or indicator) or ability to attach or include a label (e.g., a marker or indicator). Markers or indicators include, but are not limited to, for example, radioactive molecules, colorimetric molecules, and enzymatic molecules which produce detectable changes in a substrate.

In addition, an galectin-3 or biomarker protein may be detected using Mass Spectrometry such as MALDI/TOF (time-of-flight), SELDI-TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 (Hutchens & Yip) and WO 98/59361 (Hutchens & Yip). The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition, Skoog, Saunders College Publishing, Philadelphia, 1985; and Kirk-Othmer Encyclopedia of Chemical Technology, 4.^{th} ed. Vol. 15 (John Wiley & Sons, New York 1995), pp. 1071-1094.

Detection of the presence of a marker or other substances may typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample, a reference sample, and or one or more test samples may be distinguished by the presence of heavy atoms (e.g., ¹³C), optionally by using isotopically differentiated labels linked to the substrate to be detected in an array of samples, thereby permitting multiple samples to be combined and differentiated in the same mass spectrometry run.

In certain preferred embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments, the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to better quantify the amount of the biomolecule present in the first sample. In certain embodiments, the identity of the biomolecules in the first and second sample can also be determined.

### VI. Galectin-3 and Biomarker RNA Detection Techniques

Any method for qualitatively or quantitatively detecting galectin-3/biomarker RNA, e.g., mRNA, may be used.

Detection of RNA transcripts may be achieved by Northern blotting, for example, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

In certain embodiments, quantitative real-time polymerase chain reaction (qRT-PCR) is used to evaluate mRNA levels of galectin-3 (see Examples). Galectin-3/biomarker and a control mRNA, e.g., glyceraldehyde-3-phosphate dehydrogenase (GAPDH) mRNA levels may be quantitated in cancer tissue and adjacent benign tissues. For this, frozen tissues may be cut into 5 micron sections and total RNA may be extracted, e.g., by Qiagen RNeasy Mini Kit (Qiagen, Inc., Valencia, Calif.). A certain amount of RNA, e.g., five hundred nanograms of total RNA, from each tissue may be reversely transcribed by using, e.g., Qiagen Omniscript RT Kit. Two-step qRT-PCR may be performed, e.g., with the ABI TaqMan PCR reagent kit (ABI Inc, Foster City, Calif.), and galectin-3 primers and GAPDH primers, and the probes for both genes on ABI Prism 7700 system. Suitable primers that may be used are set forth in the Examples. The galectin-3/biomarker copy number may then be divided by the GAPDH copy number and multiplied by 1,000 to give a value for the particular subject. In other words, the amount of galectin-3/biomarker mRNA was normalized with the amount of GAPDH mRNA measured in the same RNA extraction to obtain a galectin-3/biomarker/GAPDH ratio. A ratio that is equal to or more than 1, e.g., about 1.1, 1.2, 1.3, 1.4, 1.5, 2, 2.5, 3, 5, 10, 30, or 100 may be considered as a high galectin-3/biomarker expression.

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

Primers that may be used for amplification of galectin-3 nucleic acid portions are set forth in the Examples.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

Another method for evaluation of galectin-3/biomarker expression is to detect gene amplification by fluorescent in situ hybridization (FISH). FISH is a technique that can directly identify a specific region of DNA or RNA in a cell and therefore enables visual determination of the galectin-3/biomarker expression in tissue samples. The FISH method has the advantages of a more objective scoring system and the presence of a built-in internal control consisting of the galectin-3/biomarker gene signals present in all non-neoplastic cells in the same sample. Fluorescence in situ hybridization is a direct in situ technique that is relatively rapid and sensitive. FISH test also can be automated. Immunohistochemistry can be combined with a FISH method when the expression level of galectin-3/biomarker is difficult to determine by immunohistochemistry alone.

Alternatively, mRNA expression can be detected on a DNA array, chip or a microarray. Oligonucleotides corresponding to the galectin-3/biomarker may be immobilized on a chip which is then hybridized with labeled nucleic acids of a test sample obtained from a patient. Positive hybridization signal can be obtained with the sample containing galectin-3/biomarker transcripts. Methods of preparing DNA arrays and their use are well known in the art. (See, for example U.S. Pat. Nos. 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA can be extracted from the biological sample to be tested, reverse transcribed, and fluorescent-labeled cDNA probes are generated. The microarrays capable of hybridizing to galectin-3/biomarker cDNA are then probed with the labeled cDNA probes, the slides scanned and fluorescence intensity measured. This intensity correlates with the hybridization intensity and expression levels.

Types of probes for detection of galectin-3/biomarker RNA include cDNA, riboprobes, synthetic oligonucleotides and genomic probes. The type of probe used may generally be dictated by the particular situation, such as riboprobes for in situ hybridization, and cDNA for Northern blotting, for example. Most preferably, the probe is directed to nucleotide regions unique to galectin-3/biomarker RNA. The probes may be as short as is required to differentially recognize galectin-3/biomarker mRNA transcripts, and may be as short as, for example, 15 bases; however, probes of at least 17 bases, more preferably 18 bases and still more preferably 20 bases are preferred. Preferably, the primers and probes hybridize specifically under stringent conditions to a DNA fragment having the nucleotide sequence corresponding to the galectin-3 gene. As herein used, the term “stringent conditions” means hybridization may occur only if there is at least 95% and preferably at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

### VII. Methods for Treating Kidney Disorder with a Galectin-3 Inhibitor

The invention provides methods for treating kidney disorder in patients with a galectin-3 inhibitor or modified pectin, e.g., GCS-100.

A. Dose and Dose Regimen

In some embodiments, the total amount of a therapeutically effective substance (galectin-3 inhibitor or modified pectin, e.g., GCS-100) in a composition to be administered (e.g., injected or intravenously infused) to a patient is one that is suitable for that patient. One of skill in the art would appreciate that different individuals may require different total amounts of the galectin-3 inhibitor or modified pectin. In some embodiments, the amount of the galectin-3 inhibitor or modified pectin is a pharmaceutically effective amount. The skilled worker would be able to determine the amount of the galectin-3 inhibitor or modified pectin in a composition needed to treat a patient based on factors such as, for example, the age, weight, and physical condition of the patient. The concentration of the galectin-3 inhibitor or modified pectin depends in part on its solubility in the intravenous administration solution and the volume of fluid that can be administered.

In certain embodiments, a galectin-3 inhibitor or modified pectin (e.g., GCS-100), is administered to the subject at a fixed dose ranging from 0.1 mg/m² to 30 mg/m². For example, a modified pectin or galectin-3 inhibitor may be administered to the subject in a fixed dose of 0.1 mg/m², 0.5 mg/m², 1 mg/m², 3 mg/m², 6 mg/m², 9 mg/m², 12 mg/m², 15 mg/m², 18 mg/m², 21 mg/m², 24 mg/m², 27 mg/m², 30 mg/m², 35 mg/m², 40 mg/m², 50 mg/m², 60 mg/m², 70 mg/m², 80 mg/m², 90 mg/m², 100 mg/m², 10 mg/m², 120 mg/m², 130 mg/m², 140 mg/m², 150 mg/m², 160 mg/m², 170 mg/m², 180 mg/m², 190 mg/m², 200 mg/m², etc. Ranges of values between any of the aforementioned recited values are also intended to be included in the scope of the invention, e.g., 0.2 mg/m², 0.6 mg/m², 1.5 mg/m², 2 mg/m², 4 mg/m², 8 mg/m², 10 mg/m², 13 mg/m², 17 mg/m², 20 mg/m², 23 mg/m², 25 mg/m², 26 mg/m², 28 mg/m², 32 mg/m², 45 mg/m², 55 mg/m², 65 mg/m², 75 mg/m², 85 mg/m², 95 mg/m², 105 mg/m², 115 mg/m², 125 mg/m², 135 mg/m², 145 mg/m², 155 mg/m², 165 mg/m², 175 mg/m², 185 mg/m², 195 mg/m², 205 mg/m², as are ranges based on the forementioned doses, e.g., 0.1-5 mg/m², 5-10 mg/m², 10-15 mg/m², 15-20 mg/m², 20-25 mg/m², 25-30 mg/m², 30-80 mg/m², 80-120 mg/m², 120-150 mg/m², 150-175 mg/m², 175-200 mg/m², The total body dose should not exceed 1 g/m² weekly or 200 mg/m² daily times 5.

In certain embodiments, a galectin-3 inhibitor or modified pectin (e.g., GCS-100), is administered to the subject at a fixed dose ranging from 1-10 mg, e.g., weekly. For example, the fixed dose may be 1 mg, 2 mg, 3 mg, 4 mg, 5 mg, 6 mg, 7 mg, 8 mg, 9 mg, or 10 mg, e.g., weekly in each case. In certain such embodiments, a modified pectin, preferably GCS-100, is administered weekly for an initial period (e.g., an induction phase, such as 1-3 months, preferably 2 months) followed by biweekly administration (e.g., a maintenance or treatment phase, such as 1-6 months, or even indefinitely) thereafter. In certain such embodiments, the fixed dose is the same throughout both phases, with only the frequency of administration varying between the two phases.

The concentration of the galectin-3 inhibitor or modified pectin in the composition administered can be at least 16 ug/ml. In some embodiments, the concentration of the galectin-3 inhibitor or modified pectin may be about 1.0 ug/ml, about 2.0 ug/ml, about 3.0 ug/ml, about 4.0 ug/ml, about 5.0 ug/ml, about 6.0 ug/ml, about 7.0 ug/ml, about 8.0 ug/ml, about 9.0 ug/ml, about 10.0 ug/ml, about 11.0 ug/mi, about 12.0 ug/ml, about 13.0 ug/ml, about 14.0 ug/ml, about 15.0 ug/ml, etc. The composition including the galectin-3 inhibitor or modified pectin can be administered at a rate sufficient to achieve an increase or modulation in one or more physiological parameters, such as glomerular filtration rate, renal vascular resistance, renal blood flow, filtration fractions, mean arterial pressure, etc., or in the levels of one or more biomarkers, as discussed herein. A patient may be coupled to a monitor that provides continuous, periodic, or occasional measurements during some or all of the course of treatment. The rate of administration may be modulated manually (e.g., by a physician or nurse) or automatically (e.g., by a medical device capable of modulating delivery of the composition in response to physiological parameters received from the monitor) to maintain the patient's physiological and/or biomarker parameters within a desired range or above or below a desired threshold or example, the rate of administration of the galectin-3 inhibitor or modified pectin may be from about 0.032 ng/kg/min to about 100 ug/kg/min in the injectable composition. In some embodiments, the rate of administration of the galectin-3 inhibitor or modified pectin may be from about 0.4 to about 45 ug/min, from about 0.12 to about 19 ug/min, from about 3.8 to about 33.8 ug/min, from about 0.16 to about 2.6 ug/min, etc. In particular embodiments, the rate of administration of the galectin-3 inhibitor or modified pectin may be about 0.032 ng/kg/min, about 0.1 ng/kg/min, about 0.32 ng/kg/min, about 1 ng/kg/min, about 1.6 ng/kg/min, about 2 ng/kg/min, about 3 ng/kg/min, about 4 ng/kg/min, about 5 ng/kg/min, about 6 ng/kg/min, about 7 ng/kg/min, about 8 ng/kg/min, about 9 ng/kg/min, about 10 ng/kg/min, about 15 ng/kg/min, about 20 ng/kg/min, about 25 ng/kg/min, about 30 ng/kg/min, about 40 ng/kg/min, about 50 ng/kg/min, about 60 ng/kg/min, about 70 ng/kg/min, about 80 ng/kg/min, about 90 ng/kg/min, about 100 ng/kg/min, about 200 ng/kg/min, about 300 ng/kg/min, about 400 ng/kg/min, about 500 ng/kg/min, about 600 ng/kg/min, about 700 ng/kg/min, about 800 ng/kg/min, about 900 ng/kg/min, about 1 ug/kg/min, about 1.1 ug/kg/min, about 1.2 ug/kg/min, about 1.3 ug/kg/min, about 1.4 ug/kg/min, about 1.5 ug/kg/min, about 1.5 ug/kg/min, about 1.6 ug/kg/min, about 1.7 ug/kg/min, about 1.8 ug/kg/min, about 1.9 ug/kg/min, about 2 ug/kg/min, about 2.1 ug/kg/min, about 2.2 ug/kg/min, about 2.3 ug/kg/min, about 2.4 ug/kg/min, about 2.5 ug/kg/min, about 2.6 ug/kg/min, about 2.7 ug/kg/min, about 2.8 ug/kg/min, about 2.9 ug/kg/min, about 3.0 ug/kg/min, about 3.1 ug/kg/min, about 3.2 ug/kg/min, about 3.3 ug/kg/min, about 3.4 ug/kg/min, about 3.5 ug/kg/min, about 3.6 ug/kg/min, about 3.7 ug/kg/min, about 3.8 ug/kg/min, about 3.9 ug/kg/min, about 4.0 ug/kg/min, about 4.1 ug/kg/min, about 4.2 ug/kg/min, about 4.3 ug/kg/min, about 4.4 ug/kg/min, about 4.5 ug/kg/min, about 4.6 ug/kg/min, about 4.7 ug/kg/min, about 4.8 ug/kg/min, about 4.9 ug/kg/min, about 5.0 ug/kg/min, about 6 ug/kg/min, about 7 ug/kg/min, about 8 ug/kg/min, about 9 ug/kg/min, about 10 ug/kg/min, about 11 ug/kg/min, about 12 ug/kg/min, about 13 ug/kg/min, about 14 ug/kg/min, about 15 ug/kg/min, about 16 ug/kg/min, about 17 ug/kg/min, about 18 ug/kg/min, about 19 ug/kg/min, about 20 ug/kg/min, about 25 ug/kg/min, about 30 ug/kg/min, about 31 ug/kg/min, about 32 ug/kg/min, about 33 ug/kg/min, about 33.8 ug/kg/min, about 34 ug/kg/min, about 35 ug/kg/min, about 40 ug/kg/min, about 45 ug/kg/min, about 50 ug/kg/min, about 55 ug/kg/min, about 60 ug/kg/min, about 65 ug/kg/min, about 70 ug/kg/min, about 75 ug/kg/min, about 80 ug/kg/min, about 85 ug/kg/min, about 90 ug/kg/min, about 95 ug/kg/min, about 100 ug/kg/min, etc.

The composition may be administered over a period of time selected from at least 8 hours; at least 24 hours; and from 8 hours to 24 hours. The composition may be administered continuously for at least 2-6 days, such as 2-11 days, continuously for 2-6 days, for 8 hours a day over a period of at least 2-6 days, such as 2-11 days. A weaning period (from several hours to several days) may be beneficial after prolonged infusion. In certain embodiments, the duration of treatment may last up to 8 consecutive weeks of dosing or until the development of dose-limiting toxicity.

B. Pharmaceutical Formulations

The compositions of the invention can be administered through any suitable route. In some embodiments, the compositions of the invention are suitable for parenteral administration. These compositions may be administered, for example, intraperitoneally, intravenously, intrarenally, or intrathecally. In some embodiments, the compositions of the invention are injected intravenously. One of skill in the art would appreciate that a method of administering a therapeutically effective substance formulation or composition of the invention would depend on factors such as the age, weight, and physical condition of the patient being treated, and the disease or condition being treated. The skilled worker would, thus, be able to select a method of administration optimal for a patient on a case-by-case basis.

The compositions may be solutions containing at least 0.5%, 1%, 5% or 10% by weight of the galectin-3 inhibitor or modified pectin, e.g., up to about 10% or 15% by weight. In certain embodiments, the modified pectin is provided as a colloidal solution in water. The size of the colloidal particles may be less than 1 μm in diameter, preferably less than about 0.65 μm, and most preferably less than about 0.2 μm.

The formulation may comprise suitable excipients including pharmaceutically acceptable buffers, stabilizers, local anesthetics, and the like that are well known in the art. For parenteral administration, an exemplary formulation may be a sterile solution or suspension; For oral dosage, a syrup, tablet or palatable solution; for topical application, a lotion, cream, spray or ointment; for intravaginal or intrarectal administration, pessaries, suppositories, creams or foams. Preferably, the route of administration is parenteral, more preferably intravenous.

In alternative embodiments, a pharmaceutical composition of the invention may be in a form adapted for oral dosage, such as for example a syrup or palatable solution; a form adapted for topical application, such as for example a cream or ointment; or a form adapted for administration by inhalation, such as for example a microcrystalline powder or a solution suitable for nebulization. Methods and means for formulating pharmaceutical ingredients for alternative routes of administration are well-known in the art, and it is to be expected that those skilled in the relevant arts can adapt these known methods to the galectin-3 inhibitors of the invention.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention may optionally be scored or prepared with coatings and shells, such as enteric coatings and other coatings well known in the pharmaceutical-formulating art. They may also be formulated so as to provide slow or controlled release of the modified therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally in a delayed manner. Examples of embedding compositions that can be used include polymeric substances and waxes. The galectin-3 inhibitor can also be in micro-encapsulated form, if appropriate, with one or more of the above-described excipients.

Liquid dosage forms for oral administration of the galectin-3 inhibitors of the invention include pharmaceutically acceptable emulsions, microemulsions, solutions, suspensions, syrups and elixirs. In addition to the galectin-3 inhibitor, the liquid dosage forms may contain inert diluents commonly used in the art, such as, for example, water or other solvents, solubilizing agents and emulsifiers.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Administration of medicament may be indicated for the treatment of mild, moderate or severe acute or chronic symptoms or for prophylactic treatment. It may be appreciated that the precise dose administered may depend on the age and condition of the patient, the particular particulate medicament used and the frequency of administration and may ultimately be at the discretion of the attendant physician. Typically, administration may occur weekly, though may occur at a regular or irregular frequency, such as daily or monthly or a combination thereof (e.g., daily for five days once a month).

Pharmaceutical compositions of this invention suitable for parenteral administration comprise a galectin-3 inhibitor of the invention in combination with one or more pharmaceutically acceptable sterile isotonic aqueous or non-aqueous solutions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Examples of pharmaceutically acceptable antioxidants include but are not limited to ascorbic acid, cysteine hydrochloride, sodium metabisulfite, sodium sulfite, ascorbyl palmitate, butylated hydroxyanisole (BHA), butylated hydroxytoluene (BHT), propyl gallate, alpha-tocopherol, and chelating agents such as citric acid, ethylenediamine tetraacetic acid (EDTA), sorbitol, tartaric acid, phosphoric acid, and the like.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

Dosage forms for the topical or transdermal administration of a compound of this invention include powders, sprays, ointments, pastes, creams, lotions, gels, solutions, patches and inhalants. The galectin-3 inhibitor may be mixed under sterile conditions with a pharmaceutically acceptable carrier, and with any preservatives, buffers, or propellants that may be required.

A pH-adjusting agent may be beneficial to adjust the pH of the compositions by including a pH-adjusting agent in the compositions of the invention. Modifying the pH of a formulation or composition may have beneficial effects on, for example, the stability or solubility of a therapeutically effective substance, or may be useful in making a formulation or composition suitable for parenteral administration. pH-adjusting agents are well known in the art. Accordingly, the pH-adjusting agents described herein are not intended to constitute an exhaustive list, but are provided merely as exemplary pH-adjusting agents that may be used in the compositions of the invention. pH-adjusting agents may include, for example, acids and bases. In some embodiments, a pH-adjusting agent includes, but is not limited to, acetic acid, hydrochloric acid, phosphoric acid, sodium hydroxide, sodium carbonate, and combinations thereof. The pH of the compositions of the invention may be any pH that provides desirable properties for the formulation or composition. Desirable properties may include, for example, therapeutically effective substance stability, increased therapeutically effective substance retention as compared to compositions at other pHs, and improved filtration efficiency. In some embodiments, the pH of the compositions of the invention may be from about 3.0 to about 9.0, e.g., from about 5.0 to about 7.0. In particular embodiments, the pH of the compositions of the invention may be 5.5-0.1, 5.6±0.1, 5.7±0.1, 5.8±0.1, 5.9±0.1, 6.0±0.1, 6.1±0.1, 6.2±0.1, 6.3±0.1, 6.4±0.1, or 6.5±0.1.

In certain embodiments, the galectin-3 inhibitor is a modified pectin which is prepared substantially ethanol-free and suitable for parenteral administration. By substantially free of ethanol, it is meant that the compositions of the invention contain less than 5% ethanol by weight. In preferred embodiments the compositions contain less than 2%, and more preferably less than 0.5% ethanol by weight. In certain embodiments, the compositions further comprise one or more pharmaceutically acceptable excipients. Such compositions include aqueous solutions of the galectin-3 inhibitor of the invention. In certain embodiments of such aqueous solutions, the pectin modification occurs at a concentration of at least 7 mg/mL, at least 10, or 15 or more mg/ml. Any of such compositions are also substantially free of organic solvents other than ethanol.

A buffer may be used to resuspend the compound in solution. In certain embodiments, a buffer may have a pKa of, for example, about 5.5, about 6.0, or about 6.5. One of skill in the art would appreciate that an appropriate buffer may be chosen for inclusion in compositions of the invention based on its pKa and other properties. Buffers are well known in the art. Accordingly, the buffers described herein are not intended to constitute an exhaustive list, but are provided merely as exemplary buffers that may be used in the compositions of the invention. In certain embodiments, a buffer may include one or more of the following: Tris, Tris HCl, potassium phosphate, sodium phosphate, sodium citrate, sodium ascorbate, combinations of sodium and potassium phosphate, Tris/Tris HCl, sodium bicarbonate, arginine phosphate, arginine hydrochloride, histidine hydrochloride, cacodylate, succinate, 2-(N-morpholino)ethanesulfonic acid (MES), maleate, bis-tris, phosphate, carbonate, and any pharmaceutically acceptable salts and/or combinations thereof.

A solubilizing agent may be added to increase the solubility of a drug or compound. In some embodiments, it may be beneficial to include a solubilizing agent to the galectin-3 inhibitor or modified pectin. Solubilizing agents may be useful for increasing the solubility of any of the components of the formulation or composition, including a therapeutically effective substance galectin-3 inhibitor or an excipient. The solubilizing agents described herein are not intended to constitute an exhaustive list, but are provided merely as exemplary solubilizing agents that may be used in the compositions of the invention. In certain embodiments, solubilizing agents include, but are not limited to, ethyl alcohol, tert-butyl alcohol, polyethylene glycol, glycerol, methylparaben, propylparaben, polyethylene glycol, polyvinyl pyrrolidone, and any pharmaceutically acceptable salts and/or combinations thereof.

A stabilizing agent may help to increase the stability of a therapeutically effective substance in compositions of the invention. This may occur by, for example, reducing degradation or preventing aggregation of a therapeutically effective substance. Without wishing to be bound by theory, mechanisms for enhancing stability may include sequestration of the therapeutically effective substance from a solvent or inhibiting free radical oxidation of the anthracycline compound. Stabilizing agents are well known in the art. Accordingly, the stabilizing agents described herein are not intended to constitute an exhaustive list, but are provided merely as exemplary stabilizing agents that may be used in the compositions of the invention. Stabilizing agents may include, but are not limited to, emulsifiers and surfactants.

A surfactant may be added to reduce the surface tension of a liquid composition. This may provide beneficial properties such as improved ease of filtration. Surfactants also may act as emulsifying agents and/or solubilizing agents. Surfactants are well known in the art. Accordingly, the surfactants described herein are not intended to constitute an exhaustive list, but are provided merely as exemplary surfactants that may be used in the compositions of the invention. Surfactants that may be included include, but are not limited to, sorbitan esters such as polysorbates (e.g., polysorbate 20 and polysorbate 80), lipopolysaccharides, polyethylene glycols (e.g., PEG 400 and PEG 3000), poloxamers (i.e., pluronics), ethylene oxides and polyethylene oxides (e.g., Triton X-100), saponins, phospholipids (e.g., lecithin), and combinations thereof.

A tonicity-adjusting reagent may be used to help make a formulation or composition suitable for administration. The tonicity of a liquid composition is an important consideration when administering the composition to a patient, for example, by parenteral administration. Tonicity-adjusting agents are well known in the art. Accordingly, the tonicity-adjusting agents described herein are not intended to constitute an exhaustive list, but are provided merely as exemplary tonicity-adjusting agents that may be used in the compositions of the invention. Tonicity-adjusting agents may be ionic or non-ionic and include, but are not limited to, inorganic salts, amino acids, carbohydrates, sugars, sugar alcohols, and carbohydrates. Exemplary inorganic salts may include sodium chloride, potassium chloride, sodium sulfate, and potassium sulfate. An exemplary amino acid is glycine. Exemplary sugars may include sugar alcohols such as glycerol, propylene glycol, glucose, sucrose, lactose, and mannitol.

B. Articles of Manufacture and Kits

The invention also provides a packaged pharmaceutical composition wherein the galectin-3 inhibitor or modified pectin, e.g., GCS-100, is packaged within a kit or an article of manufacture. The kit or article of manufacture of the invention may contain materials useful for the treatment, including the improvement, and/or remission, prevention and/or diagnosis or monitoring of kidney disorder. The kit or article of manufacture may comprise a container and a label or package insert or printed material on or associated with the container which provides information regarding use of the galectin-3 inhibitor or modified pectin for the treatment of kidney disorder.

In certain embodiments, the invention provides an article of manufacture comprising a galectin-3 inhibitor and a package insert, wherein the package insert indicates that the galectin-3 inhibitor may be used to treat kidney disorder in patients who have an eGFR in the range of about 15-44 mL/min/1.73 m².

The term “package insert” is used to refer to instructions customarily included in commercial packages of therapeutic products, that contain information about the indications, usage, dosage, administration, contraindications and/or warnings concerning the use of such therapeutic products.

In certain embodiments, the article of manufacture of the invention comprises (a) a first container holding a composition comprising a galectin-3 inhibitor or modified pectin; and (b) a package insert indicating how the galectin-3 inhibitor or modified pectin may be administered to a patient, as discussed herein. In preferred embodiments, the label or package insert indicates that the galectin-3 inhibitor or modified pectin (e.g. GCS-100), is used for treating a kidney disorder. In certain embodiments, the invention features a kit comprising a sufficient number of containers to provide both loading and maintenance doses of the galectin-3 inhibitor or modified pectin. For example, the kit may contain containers containing about 1.5 and 30 mg/m², or amounts ranging from 0.1-5 mg/m², 5-10 mg/m², 10-15 mg/m², 15-20 mg/m², 20-25 mg/m², 25-30 mg/m², 30-80 mg/m², 80-120 mg/m², 120-150 mg/m², 150-175 mg/m², 175-200 mg/m², of modified pectin for intravenous injection. The containers each containing the galectin-3 inhibitor or modified pectin (e.g. GCS-100) could, for example, provide enough modified pectin to be administered intravenously once weekly for up to 8 consecutive weeks, or at another suitable frequency such as daily or monthly.

Suitable containers for the galectin-3 inhibitor or modified pectin (e.g. GCS-100), include, for example, bottles, vials, syringes, including preloaded/pre-filled syringes, pens, including autoinjector pens, etc. The containers may be formed from a variety of materials such as glass or plastic. The container holds a composition which is by itself or when combined with another composition effective for treating, preventing and/or diagnosing the condition and may have a sterile access port.

In certain embodiments, the pharmaceutical compositions and associated articles of manufacture are useful in treating certain patient populations who may respond favorably to the modified pectin. For example, the modified pectin, e.g., GCS-100, may be used to treat kidney disorder in patients who have been unresponsive or intolerant to oral antibiotics or medication for treatment for their kidney disorder.

In certain embodiments, the pharmaceutical compositions and/or associated articles of manufacture may provide a dose suitable for administration of the therapeutic agent for the treatment of a kidney disorder. In certain embodiments, the article includes a loading dose of about 1.5 mg/m² to be administered at the outset of therapy. In certain embodiments, the article includes a maintenance dose of about 0.5 mg/m², e.g., for a number of weeks thereafter, such as starting from week 4. For example, a kit of the invention may include a loading dose and one or more maintenance doses.

In other embodiments, the article provides a galectin-3 inhibitor or modified pectin (e.g. GCS-100) suitable for subcutaneous injection.

In certain embodiments of the invention, the kit comprises a galectin-3 inhibitor or modified pectin, a second pharmaceutical composition comprising an additional therapeutic agent, and optionally instructions for administration of both agents for the treatment of kidney disorder. The instructions may describe how, e.g., subcutaneously or intravenously, and when, e.g., at week 0, week 2, and weekly or biweekly thereafter, doses of modified pectin and/or the additional therapeutic agent shall be administered to a subject for treatment.

In certain embodiments, the kits contain a pharmaceutical composition comprising a galectin-3 inhibitor or modified pectin and a pharmaceutically acceptable carrier and one or more additional pharmaceutical compositions each comprising a drug useful for treating a kidney disorder (such as CKD or NASH) or a symptom thereof and a pharmaceutically acceptable carrier. Alternatively, the kit comprises a single pharmaceutical composition comprising a galectin-3 inhibitor (such as a modified pectin), one or more drugs useful for treating a kidney disorder (such as CKD or NASH), and a pharmaceutically acceptable carrier.

In another aspect, the invention provides a pharmaceutical package, comprising a vial or ampoule containing a galectin-3 inhibitor according to the invention in the form of a reconstitutable powder or a solution suitable for injection or infusion, optionally together with instructions for administering the composition to a patient suffering from nephrotoxicity. Instructions include but are not limited to written and/or pictorial descriptions of: the active ingredient, directions for diluting the composition to a concentration suitable for administration, suitable indications, suitable dosage regimens, contraindications, drug interactions, and any adverse side-effects noted in the course of clinical trials.

In alternative embodiments, the pharmaceutical package may comprise a plastic bag containing from 100 mL to 2 L of a pharmaceutical composition of the invention, in the form of a solution suitable for intravenous administration, optionally together with instructions as described above.

C. Additional Therapeutic Agents

Galectin-3 inhibitors or modified pectins, including GCS-100, may be used in the methods of the invention either alone or in combination with an additional therapeutic agent, said additional agent being selected by the skilled artisan for its intended purpose. For example, the additional agent can be a therapeutic agent art recognized as being useful to treat the disease or condition being treated by the galectin-3 inhibitor or modified pectins.

It should further be understood that the combinations which are to be included within this invention are those combinations useful for their intended purpose. The therapeutic agents set forth below are illustrative for purposes and not intended to be limited. The combinations, which are part of this invention, can be the galectin-3 inhibitor or modified pectin and at least one additional agent selected from the lists below. The combination can also include more than one additional agent, e.g., two or three additional therapeutic agents if the combination is such that the formed composition can perform its intended function. Modified pectins or galectin-3 inhibitors described herein may be used in combination with additional therapeutic agents for the treatment of cancer, cardiovascular disease, inflammation, fibrosis, and renal injury, which may act parallel to, dependent on or in concert with modified pectin function. The modified pectins used in the invention may also be combined with one or more therapeutic agents, such as methotrexate, mesalazine, olsalazine, chloroquine, hydroxychloroquine, pencillamine, aurothiomalate (intramuscular or oral), cochicine, beta-2 adrenoreceptor agonists (salbutamol, terbutaline, salmeterol), xanthines (theophylline, aminophylline), cromoglycate, nedocromil, ketotifen, ipratropium, oxitropium, cyclosporin, FK506, rapamycin, mycophenolate mofetil, leflunomide, NSAIDs (for example, ibuprofen), corticosteroids (such as prednisolone, methylprednisolone, and methylprednisolone acetate), phosphodiesterase inhibitors, adensosine agonists, antithrombotic agents, complement inhibitors, adrenergic agents, agents which interfere with signaling by proinflammatory cytokines such as TNFα or IL1 (e.g. IRAK, NIK, IKK, p38 or MAP kinase inhibitors), IL-1-converting enzyme inhibitors, TNFa converting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75 TNFRiγG (Enbrel™ and p55 TNFRiγG (Lenercept)), siL-1RI, siL-1RII, siL-6R), anti-inflammatory cytokines (e.g., IL-4, IL-10, IL-12, IL-13 and TGFβ), celecoxib, folic acid, hydroxychloroquine sulfate, rofecoxib, etanercept, infliximab, naproxen, valdecoxib, meloxicam, gold sodium thiomalate, aspirin, triamcinolone acetonide, propoxyphene napsylate, folate, nabumetone, diclofenac, piroxicam, etodolac, diclofenac sodium, oxaprozin, oxycodone, hydrocodone bitartrate, diclofenac sodium, misoprostol, fentanyl, anakinra, tramadol, salsalate, sulindac, cyanocobalamin, folacin, pyridoxine, acetaminophen, alendronate sodium, prednisolone, morphine sulfate, lidocaine, indomethacin, glucosamine sulfate, chondroitin, amitriptyline, sulfadiazine, olopatadine, omeprazole, cyclophosphamide, rituximab, IL-1 TRAP, MRA, CTLA4-IG, IL-18 BP, anti-IL-18, Anti-IL15, BIRB-796, SCI0-469, VX-702, AMG-548, VX-740, Roflumilast, IC-485, PSORIASIS C-801, and Mesopram.

Non-limiting examples of therapeutic agents for kidney disorder with which modified pectins or other galectin-3 inhibitors can be combined include the following: antiseptic and antiperspirant agents (e.g., 6.25% aluminum chloride hexahydrate in absolute ethanol), anti-inflammatory or anti-antiandrogen therapy such as tetracycline, intralesional triamcinolone, or finasteride. The galectin-3 inhibitors or modified pectins may also be combined with agents, such as methotrexate, cyclosporin, FK506, rapamycin, mycophenolate mofetil, leflunomide, NSAIDs (for example, ibuprofen), corticosteroids such as prednisolone, phosphodiesterase inhibitors, adenosine agonists, antithrombotic agents, complement inhibitors, adrenergic agents, agents which interfere with signaling by proinflammatory cytokines such as TNFα or IL-1 (e.g., IRAK, NIK, IKK, p38 or MAP kinase inhibitors), IL-1β converting enzyme inhibitors, TNFα converting enzyme inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors, siL-1RI, siL-1RII, siL-6R) and anti-inflammatory cytokines (e.g., IL-4, IL-10, IL-12, IL-13 and TGFβ).

Additional examples of therapeutic agents for kidney disorder with which a modified pectin can be combined include the following: D2E7 (PCT Publication No. WO 97/29131; Humira®), Ca2 (Remicade®), TNFR-Ig constructs, (p75 TNFRiγG (Enbrel™) and p55 TNFRiγG (Lenercept) inhibitors and PDE4 inhibitors. Galectin-3 inhibitors or modified pectins can be combined with corticosteroids, for example, budenoside and dexamethasone. Galectin-3 inhibitors or modified pectins may also be combined with agents such as sulfasalazine, 5-aminosalicylic acid, and olsalazine, and agents which interfere with synthesis or action of proinflammatory cytokines such as IL1, for example, IL-1β converting enzyme inhibitors and IL-1rα. Galectin-3 inhibitors or modified pectins may also be used with T cell signaling inhibitors, for example, tyrosine kinase inhibitors 6-mercaptopurines. Galectin-3 inhibitors or modified pectins can be combined with IL-12. Galectin-3 inhibitors or modified pectins can be combined with mesalamine, prednisone, azathioprine, mercaptopurine, infliximab, methylprednisolone, diphenoxylate/atrop sulfate, loperamide hydrochloride, methotrexate, omeprazole, folate, ciprofloxacin, hydrocodone bitartrate, tetracycline hydrochloride, fluocinonide, metronidazole, thimerosal, cholestyramine, ciprofloxacin hydrochloride, hyoscyamine sulfate, meperidine hydrochloride, midazolam hydrochloride, oxycodone, promethazine hydrochloride, sodium phosphate, sulfamethoxazole I trimethoprim, celecoxib, polycarbophil, propoxyphene napsylate, hydrocortisone, multivitamins, balsalazide disodium, codeine phosphate, colesevelam hcl, cyanocobalamin, folic acid, levofloxacin, methylprednisolone, natalizumab and interferon-gamma. The galectin-3 inhibitors or modified pectins may also be combined with agents, such as alemtuzumab, dronabinol, daclizumab, mitoxantrone, xaliproden hydrochloride, fampridine, glatiramer acetate, natalizumab, sinnabidol, a-immunokine NNS03, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, AntegranELAN/Biogen), interferon gamma antagonists, IL-4 agonists, and the humanized IL-6 antibody tocilizumab.

In certain embodiments, the galectin-3 inhibitors or modified pectins may be combined with anti-viral or bacterial agents known in the art to treat infection. The term, “antibiotic,” as used herein, refers to a chemical substance that inhibits the growth of, or kills, microorganisms. Encompassed by this term are antibiotics produced by a microorganism, as well as synthetic antibiotics (e.g. analogs) known in the art. Antibiotics include, but are not limited to, clarithromycin (Biaxin®), ciprofloxacin (Cipro®), and metronidazole (Flagyl®).

In certain embodiments, the galectin-3 inhibitors or modified pectins may be combined with a chemotherapeutic agent that may cause nephrotoxicity. Alternatively, the galectin-3 inhibitors may be combined with therapies that may cause renal toxicity other than chemotherapeutics, or in response to conditions such as drug abuse or exposure to heavy metals, which are also nephrotoxic.

Cancer therapy agents associated with nephrotoxicity include alkylating agents such as AZQ (diaziquone), Cisplatin, Cisplatin analogs, Ifosfamide, nitrosoureas; antitumor antibiotics such as Mitomycin C and Plicamycin; antimetabolites such as 5-azacytidine and Methotrexate; biologic agents such as Interferon and Interleukin-2 and other drugs such as gallium nitrate, Cyclosporine and Tacrolimus. The chemotherapeutic agent may be selected from platinum complexes, Cisplatin, Oxaliplatin, Carboplatin, Nedaplatin, Satraplatin, BBR3464, or ZD0473. In certain embodiments, the galactin-3 inhibitor is administered with a chemotherapeutic or immunosuppressant selected from Cisplatin, Methotrexate, Mitomycin, Cyclosporine, Ifosfamide, and Zoledronic acid.

In certain embodiments, the galactin-3 inhibitor is combined with a nephrotoxic drug other than a chemotherapeutic, selected from antibiotics, immunosuppressants, antihyperlipidemics, ACE inhibitors, NSAIDs, and Aspirin. Antibiotics may be selected from aminoglycosides, sulfonamides, Amphotericin B, Foscarnet, quinolones (e.g., Ciprofloxacin, Levofloxacin), Rifampin, Tetracycline, Acyclovir, Pentamidine or Vancomycin. In certain embodiments, the method comprises administering a galectin-3 inhibitors or modified pectins conjointly with two or more nephrotoxic therapies such as a chemotherapeutic and an antibiotic. The method of treating kidney disorder may further include administering an additional therapeutic agent such as an anti-inflammatory drug or an antioxidant. In certain embodiments, an antioxidant may be selected from Allopurinol, Ebselen, Erdosteine, Edaravone, N-acetylcysteine, Silymarin, Naringernin, vitamin C and vitamin E. In certain embodiments, the anti-inflammatory agent is selected from salicylates.

The composition including the galectin-3 inhibitor or modified pectin may be administered in combination with additional pharmaceutical agents to facilitate improved renal function. In some embodiments, the additional pharmaceutical agent is albumin, since expansion of the volume of plasma with albumin given intravenously has shown to improve renal function in patients with hepatorenal syndrome. The quantity of the additional pharmaceutical agent administered may vary depending on the cumulative therapeutic effect of the treatment including the galectin-3 inhibitor or modified pectin and the additional pharmaceutical agent. For example, the quantity of albumin administered may be 1 gram of albumin per kilogram of body weight given intravenously on the first day, followed by 20 to 40 grams daily. Yet other additional pharmaceutical agents may be any one or more of midodrine, octreotide, somatostatin, vasopressin analogue ornipressin, terlipressin, pentoxifylline, acetylcysteine, norepinephrine, misoprostol, etc. In some embodiments, other natriuretic peptides may also be used in combination with the galectin-3 inhibitor or modified pectin therapeutic to remedy the impairment of sodium excretion associated with diseases discussed above. For example, natriuretic peptides may include any type of atrial natriuretic peptide (ANP), brain natriuretic peptide (BNP), C-type natriuretic peptide (CNP), and/or dendroaspis natriuretic peptide, etc. Several diuretic compounds may be used in combination with the galectin-3 inhibitor or modified pectin to induce urine output. For example any one or more of the xanthines such as caffeine, theophylline, theobromine: thiazides such as bendroflumethiazide, hydrochlorothiazide; potassium-sparing diuretics such as amiloride, spironolactone, triamterene, potassium canrenoate; osmotic diuretics such as glucose (especially in uncontrolled diabetes), mannitol; loop diuretics such as bumetanide, ethacrynic acid, furosemide, torsemide; carbonic anhydrase inhibitors such as acetazolamide and dorzolamide; Na—H exchanger antagonists such as dopamine; aquaretics such as goldenrod, juniper; arginine vasopressin receptor 2 antagonists such as amphotericin B, lithium citrate; acidifying salts such as CaCl₂, NH₄Cl; etc. may be used in combination with the galectin-3 inhibitor or modified pectin to treat the patient. The list of additional pharmaceutical agents described above is merely illustrative and may include any other pharmaceutical agents that may be useful for the treatment of renal failure associated with any of the kidney disorders discussed herein.

1. Conjoint Administration

The conjoint administration of the galectin-3 inhibitor and an additional therapeutic agent may involve concurrent administration. In particular embodiments, conjoint administration involves administration of the two agents within about 10 min, about 20 min, or about 30 minutes of each other. In exemplary embodiments, the galectin-3 inhibitor is administered in an overlapping fashion with the additional therapeutic, e.g., the additional therapeutic is administered intravenously and the galectin-3 inhibitor is administered orally during the course of the intravenous dosing.

The galectin-3 inhibitor may be administered subsequent to administration of the additional therapeutic agent. The galectin-3 inhibitor may be administered immediately after the additional therapeutic agent or within, for example, 1 hour, 2 hours, 4 hours, 6 hours or 12 hours. In other embodiments, the additional therapeutic agent may be administered subsequent to the galectin-3 inhibitor. The additional therapeutic agent may be administered immediately after the galectin-3 inhibitor or within, for example, 1 hour, 2 hours, 4 hours, 6 hours or 12 hours.

The galectin-3 inhibitor may be administered by any suitable manner in order to contact the kidney and accumulate sufficient quantities to prevent or treat renal disorder. A galectin-3 inhibitor or combination therapeutics containing a galectin-3 inhibitor may be administered orally, parenterally by intravenous injection, transdermally, by pulmonary inhalation, by intravaginal or intrarectal insertion, by subcutaneous implantation, intramuscular injection or by injection directly into an affected tissue, as for example by injection into a tumor site. In some instances the materials may be applied topically at the time surgery is carried out.

The materials are formulated to suit the desired route of administration. The galectin-3 inhibitor and any additional therapeutic agent may each be formulated in ways to facilitate administration. For example, the combination therapy may be formulated for intravenous administration while the galectin-3 inhibitor may be formulated for nebulization. The following discussion of formulation may be applied to the individual formulation of the combination therapy or galectin-3 inhibitor or combination of the two.

The galectin-3 inhibitor need not be administered in the same manner as the other combination therapy. For example, the galectin-3 inhibitor may be administered orally while the additional therapeutic agent is administered intravenously. In addition, the galectin-3 inhibitor may be administered, before, during or after the administration of the combination therapy, such as before the administration of the combination therapy. In preferred embodiments, the galectin-3 inhibitor is administered in a manner to accumulate an effective concentration of the galectin-3 inhibitor in the kidneys. Any one or more of the above-mentioned therapeutic agents, alone or in combination, can be administered to a subject suffering from kidney disorder, in combination with the galectin-3 inhibitors or modified pectins, e.g., using a multiple variable dose treatment regimen.

The method of treating a kidney disorder may further comprise hydrating the patient with saline before, during, and/or after conjoint administration of the additional therapeutic agent and galectin-3 inhibitor.

In some embodiments, any one of the above-mentioned therapeutic agents, alone or in combination therewith, can be administered to a subject suffering from kidney disorder in addition to a therapeutic agent used to treat cancer, cardiovascular disease, inflammation, etc. It should be understood that the additional therapeutic agents can be used in combination therapy as described above, but also may be used in other indications described herein wherein a beneficial effect is desired. The combination of agents used in the methods and pharmaceutical compositions described herein may have a therapeutic additive or synergistic effect on the condition(s) or disease(s) targeted for treatment. The combination of agents used within the methods or pharmaceutical compositions described herein also may reduce a detrimental effect associated with at least one of the agents when administered alone or without the other agent(s) of the particular pharmaceutical composition. For example, the toxicity of side effects of one agent may be attenuated by another agent of the composition, thus allowing a higher dosage, improving patient compliance, and/or improving therapeutic outcome. The additive or synergistic effects, benefits, and advantages of the compositions apply to classes of therapeutic agents, either structural or functional classes, or to individual compounds themselves.

### VIII. Efficacy of Galectin Inhibitors and Modified Pectins

The invention also provides methods for assessing the effects of a galectin-3 inhibitor or modified pectin in a subject. Such methods may be used to determine the efficacy of a galectin-3 inhibitor or modified pectin, or to adjust a patient's dosage in response to the measured effects. Using the methods described herein, the effects of a galectin-3 inhibitor or modified pectin may be determined or confirmed, and, optionally, used in the method of treating kidney disorder.

In certain embodiments, the invention provides a method for determining the efficacy of a galectin-3 inhibitor or modified pectin, including a GCS-100, for treating kidney disorder in a subject, using the change in baseline eGFR to determine efficacy. In certain embodiments, the efficacy of a galectin-3 inhibitor or modified pectin, including GCS-100, for treating kidney disorder in a subject is assessed by detecting a change in galectin-3 levels and/or activity, with a reduction in the level of galectin-3 being indicative of a desirable result. Other suitable markers include cystatin C, creatinine, BUN, plasma mitogen, potassium, uric acid, urea, and other markers of kidney function and/or damage.

In certain embodiments, the invention provides a method of treating kidney disorder in a subject, comprising administering a galectin-3 inhibitor or modified pectin, e.g., GCS-100, to the subject such that kidney disorder is treated, e.g., wherein the galectin-3 inhibitor or modified pectin achieves a statistically significant clinical response within a patient or patient population.

In certain embodiments, the methods of the invention are used to determine whether a dose of galectin-3 inhibitor or modified pectin is an effective dose of galectin-3 inhibitor modified pectin with respect to a patient who has been treated with the galectin-3 or modified pectin.

In certain embodiments, the methods of the invention comprise administering the galectin-3 inhibitor or modified pectin to a patient and determining the efficacy of the modified pectin by determining changes, improvements, measurements, etc., eGFR, galectin-3, biomarker, serum levels, of the patient (e.g., relative to a pretreatment condition of the patient, to a predetermined desired condition or standard, or to a condition of an untreated patient or a patient treated with placebo).

A method for determining efficacy may comprise assessing the effect on a subject who has kidney disorder of a dosage regimen comprising a galectin-3 inhibitor or modified pectin in order to determine whether the galectin-3 inhibitor or modified pectin is an effective therapy or whether a change in dosage would be desirable.

The Examples and discoveries described herein are representative of a modified pectin, GCS-100, which is effective for treating kidney disorder. As such, the studies and results described in the Examples section herein may be used as a guideline for using a galectin-3 inhibitor or modified pectin for the treatment of kidney disorder.

Other embodiments of the present invention are described in the following Examples. The present invention is further illustrated by the following examples which should not be construed as limiting in any way.

### EXEMPLIFICATIONS

### Galectin-3 Inhibitor

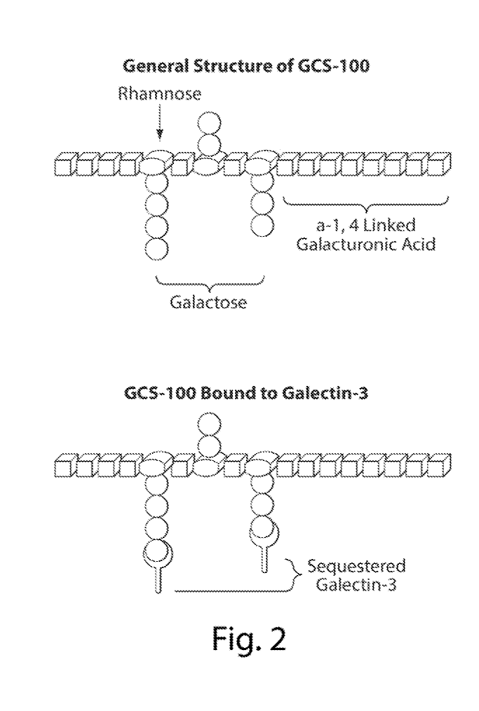

GCS-100 is a complex polysaccharide that has the ability to bind to and potentially block the effects of galectin-3. GCS-100 is a derivative of pectin, a naturally occurring polysaccharide found in the structure of various plants, including the pulp and peel of citrus fruits. Pectin is composed of several types of sugars arranged in a complex polymeric configuration with multiple side branches. In particular, pectins have multiple side-branches containing the sugar 3-galactose which is recognized by the carbohydrate binding domain of galectin-3. Thus, GCS-100 is able to bind to and sequester multiple molecules of extracellular (circulating) galectin-3 (FIG. 2). Additionally, because of its high average molecular weight, GCS-100 resides in the body for an extended period (half-life of approximately 30 hours), increasing the time to interact with and sequester circulating galectin-3.

### Example 1

### Summary of GCS-100 Pharmacology Studies

GCS-100 has been studied in a fibrotic, pro-inflammatory mouse model of fatty liver disease known as non-alcoholic steatohepatitis (NASH). In this model, NASH is established in mice by a single subcutaneous injection of streptozotocin after birth, followed by the feeding of a high-fat diet ad libitum after 4 weeks of age. NASH develops at about Week 7 with evidence of fibrosis at Week 9 and liver nodule formation at Week 11-12. In the present study, mice were randomized at 9 weeks of age into three groups treated intravenously with inactive placebo (control), 1 mg/kg GCS-100, or 25 mg/kg GCS-100. All animals received their respective administrations three times per week during Weeks 9-12. At the end of Week 12, blood and tissue samples were collected and analyzed for liver enzymes, non-alcoholic fatty liver disease (NAFLD) activity score, and fibrosis.

Overall, treatment with GCS-100 in NASH mice was well tolerated and resulted in decreased plasma ALT. No effect was observed on blood glucose levels. Histological analysis showed a significant improvement in NAFLD score with decreased micro- and macro-vesicular fat deposition, hepatocyte ballooning and inflammatory cell infiltration. A decrease in hydroxyproline was observed and a significant decrease in fibrosis, as measured by Sirius red staining, was also observed. This study demonstrates GCS-100 is effective at reducing fibrosis.

### Example 2

### Use of GCS-100 in Treatment of Patients with Chronic Kidney Disease

In order to achieve the desired therapeutic effect, it is helpful to achieve a circulating GCS-100 concentration sufficient to bind to and neutralize plasma galectin-3 at an effective level over an extended period. The average concentration of circulating galectin-3 in ESRD patients is about 64 ng/mL, which is equal to 2.21×10⁻⁶ μmol galectin-3/mL plasma (de Boer et. al., 2011).

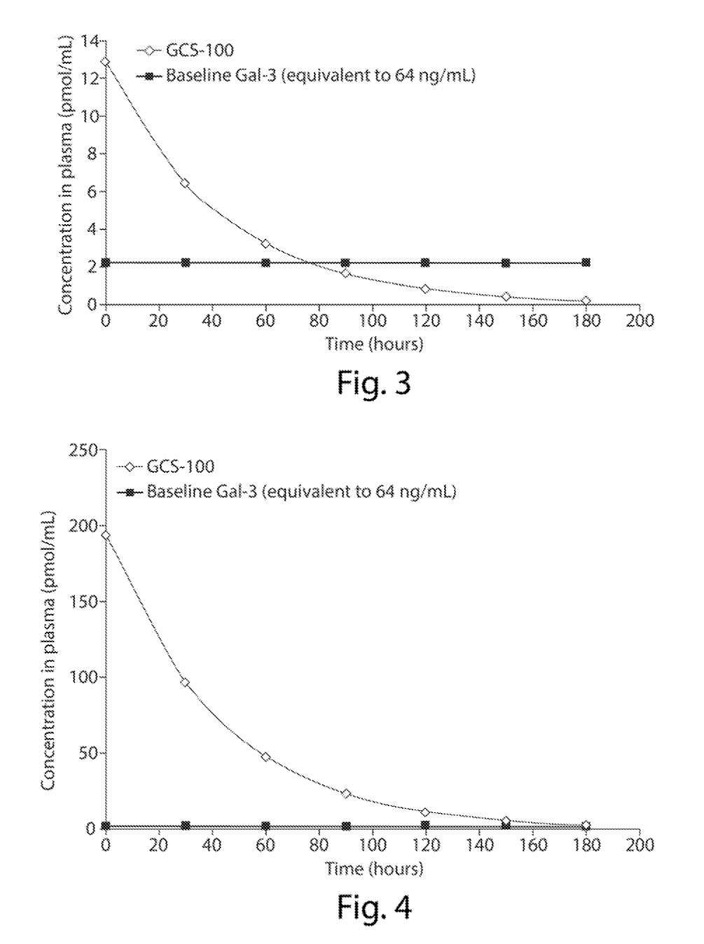

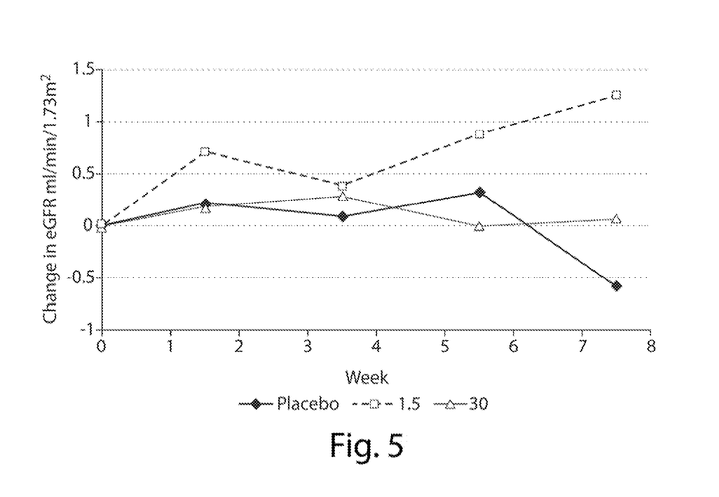

Based on human pharmacokinetic data, a single 1.5 mg/m² dose of GCS-100 is expected to result in a starting plasma concentration in excess of the expected galectin-3 concentration. At this dose on a molar basis, GCS-100 is about 6-fold more concentrated than circulating galectin-3 at the Cₘₐₓ for GCS-100. The approximate average half-life of GCS-100 in plasma is 30 hours, thus the level of GCS-100 would fall below this baseline prior to the next treatment (FIG. 3).

Similarly, a single 30 mg/m² dose of GCS-100 is expected to result in a starting plasma concentration in excess of the expected galectin-3 concentration. At this dose on a molar basis, GCS-100 is about 160-fold more concentrated than circulating galectin-3 at the Cₘₐₓ for GCS-100 and the plasma concentration of GCS-100 may not fall below this baseline prior to the next treatment.

Based on the preceding rationale, the dose groups and dosing schedule used for this study was expected to allow for effective galectin-3 inhibition, while being well tolerated.

Study Drug Administration and Dosage Schedule

Patients assigned to treatment groups received placebo or GCS-100 on Days 1, 8, 15, 22, 29, 36, 43, and 50. The amount (in mg) of GCS-100 to be administered was determined based on body surface area, calculated based on body weight and height using Formula III or IV below.

$$\begin{matrix}BSA = \sqrt{\frac{Ht ( inches ) \times Wt ( lbs )}{3131}} \\ or & Formula III \\ BSA = \sqrt{\frac{Ht ( cm ) \times Wt ( kg )}{3600}} & Formula IV\end{matrix}$$

Dosing Regimen

Patients were dosed once weekly for 8 weeks. The study drug dose for each patient was calculated on Day 1.

Treatment

Patients were randomly assigned to receive placebo (0.9% Sodium Chloride Injection, USP), 1.5 mg/m² GCS-100, or 30 mg/m² GCS-100. Placebo and GCS-100 were administered as IV infusions once weekly for 8 weeks.

Tables 3-4 show the mean change of GFR from baseline to the average of Day 50 and Day 57 for patients injected with 30 mg/m² (Table 3) and 1.5 mg/m² (Table 4). Tables 5-8 show the change in in baseline GFR, BUN, uric acid, and galectin-3 in patients administered with 1.5 mg/m of GCS-100, and 30 mg/m² of GCS-100.

TABLE 3
Summary of GFR change in patients administered
30 mg/m² of GCS-100 versus placebo
GFR change High dose vs Placebo
t-Test: Two-Sample Assuming Unequal Variances

| | Placebo | High Dose |
|---|---|---|
| Mean | −0.575 | 0.055555556 |
| Variance | 8.391666667 | 21.88253968 |
| Observations | 40 | 36 |
| Hypothesized Mean Di [symbol] | 0 | |
| df | 57 | |
| t Stat | −0.697336334 | |
| P(T <= t) one-tail | 0.244213488 | |
| t Critical one-tail | 1.672028888 | |
| P(T <= t) two-tail | 0.488426976 | |
| t Critical two-tail | 2.002465459 | |

[symbol]

indicates data missing or illegible when filed

TABLE 4
Summary of GFR change in patients administered
1.5 mg/m² of GCS-100 versus placebo
GFR change Low dose vs Placebo
t-Test: Two-Sample Assuming Unequal Variances

| | Placebo | Low dose |
|---|---|---|
| Mean | −0.575 | 1.256097561 |
| Variance | 8.391666667 | 24.32652439 |
| Observations | 40 | 41 |
| Hypothesized Mean Di [symbol] | 0 | |
| df | 65 | |
| t Stat | −2.043246906 | |
| P(T <= t) one-tail | 0.022542995 | |
| t Critical one-tail | 1.668635976 | |
| P(T <= t) two-tail | 0.045085991 | |
| t Critical two-tail | 1.997137908 | |

[symbol]

indicates data missing or illegible when filed

TABLE 5
Change in baseline eGFR (mL/min/1.73 m²) in patients administered
placebo, 1.5 mg/m² of GCS-100, and 30 mg/m² of GCS-100.

| Placebo | Low | high |
|---|---|---|
| −0.58 | 1.26 | 0.06 |

TABLE 6
Change in baseline BUN (mg/dL) in patients administered placebo,
1.5 mg/m² of GCS-100, and 30 mg/m² of GCS-100. Data presented
is for subjects with elevated (abnormal) levels at baseline.

| BUN | Change |
|---|---|
| Placebo | −0.74 |
| Low | −3.93 |
| High | 0.34 |

TABLE 7
Change in baseline uric acid (mg/dL) in patients administered
with placebo, 1.5 mg/m² of GCS-100, and 30 mg/m² of
GCS-100. Data presented is for subjects with elevated
(abnormal) levels at baseline.

| BUN | Change |
|---|---|
| Placebo | −0.55 |
| Low | −1.64 |
| High | 0.13 |

TABLE 8
Change in baseline galectin-3 (ng/mL) in patients administered
with placebo, 1.5 mg/m² of GCS-100, and 30 mg/m² of GCS-100.

| BUN | Change |
|---|---|
| Placebo | 1.03 |
| Low | −0.88 |
| High | 1.29 |

TABLE 9
Change in baseline potassium in patients administered
with placebo and 1.5 mg/m² of GCS-100.

| | Placebo | 1.5 mg/m² |
|---|---|---|
| n | 40 | 41 |
| Baseline | 4.42 | 4.49 |
| Average | 4.45 | 4.37 |
| day 50/57 | | |
| Change | 0.03 | −0.12 |
| StDev | 0.33 | 0.4 |
| P-value (ANOVA) | | 0.07 |

### Example 3

### Phase 2 Study of GCS-100 in Chronic Kidney Disease (CKD) Patients

A multicenter, randomized, blinded, placebo-controlled, Phase 2 study in 121 advanced CKD patients was performed. The phase 2 study met its primary efficacy endpoint of a statistically significant improvement in kidney function. Specifically, GCS-100, at a dose of 1.5 mg/m², led to a statistically significant (p=0.045) improvement in estimated glomerular filtration rate (eGFR) versus placebo after 8 weeks of dosing. This improvement, compared to placebo, was maintained 5 weeks following the completion of dosing (p=0.07). No statistically significant improvement in eGFR was observed in the 30 mg/m² dose group.

TABLE 10
Change in baseline eGFR (mL/min/1.73 m²) in patients
administered placebo, 1.5 mg/m² of GCS-100, and 30
mg/m² of GCS-100 in Phase 2 study.

| | Placebo | 1.5 mg/m² | 30 mg/m² |
|---|---|---|---|
| Dose Group | (n = 40) | (n = 41) | (n = 36) |
| Change in eGFR at | −0.6 | 1.3 | 0.1 |
| Week 8 | | | |
| (mL/min/1.73 m²) | | | |
| P Value (vs. Placebo) | | 0.045 | NS |
| Change in eGFR at | −1.5 | 0.2 | 0.0 |
| Week 12 | | | |
| (mL/min/1.73 m²) | | | |
| P Value (vs. Placebo) | | 0.07 | NS |

GCS-100's effect on eGFR was more pronounced (p=0.029) in the prospectively defined subset of patients with diabetic etiology. Analysis of this subset was predefined based on the observation that galectin-3 is elevated in the kidneys of diabetic CKD patients, and the level of galectin-3 correlates with proteinuria (a marker of kidney health) in these patients.

TABLE 11
Change in baseline eGFR (mL/min/1.73 m²) in diabetic
patients administered placebo, 1.5 mg/m² of GCS-100, and
30 mg/m² of GCS-100 in Phase 2 study.

| | Placebo | 1.5 mg/m² | 30 mg/m² |
|---|---|---|---|
| Dose Group | (n = 29) | (n = 24) | (n = 26) |
| Change in eGFR at | −0.5 | 2.3 | −0.3 |
| Week 8 | | | |
| (mL/min/1.73 m²) | | | |
| P Value (vs. Placebo) | | 0.029 | NS |

GCS-100 was well-tolerated. There were no serious adverse events, no Grade 3/4 adverse events and no early study discontinuations in the 1.5 mg/m² group. There was no observed effect on blood pressure in any dose group.

An extension study was conducted in which patients from the Phase 2 study were re-randomized to receive either 1.5 or 30 mg/m² of GCS-100 (complete data available through week 16). Of the 93 patients enrolled in total, 33 patients had previously received placebo in the Phase 2 study before being treated with GCS-100 in the extension study. This group, which represents a set of patients receiving GCS-100 for the first time, was analyzed for efficacy. Consistent with the blinded Phase 2 results, the 1.5 mg/m² group experienced a significant improvement in eGFR. This was observed when comparing these patients' responses to both: (1) the response in the parallel randomized group receiving 30 mg/m² (p=0.04); and (2) the previous response to placebo in the blinded Phase 2 study for placebo-treated patients enrolled in the extension study (p=0.02).

TABLE 12
Change in baseline eGFR (mL/min/1.73 m²) in Phase
2 patients administered placebo, 1.5 mg/m² of GCS-100, and
30 mg/m² of GCS-100 in extension study.

| | Placebo* | 1.5 mg/m² | 30 mg/m² |
|---|---|---|---|
| Dose Group | (n = 33) | (n = 20) | (n = 13) |
| Change in eGFR at | −2.0 | 1.3 | −1.8 |
| Week 16 | | | |
| (mL/min/1.73 m²) | | | |
| P Value (vs. Placebo) | | 0.02 | NS |
| P Value (vs. 30 mg/m² | | 0.04 | |
| Dose Group) | | | |

*Previous response to placebo in the blinded Phase 2 study for placebo-treated patients enrolled in the extension study (baseline to week 12)

### REFERENCES

All publications and patents mentioned herein, including those references listed below, are hereby incorporated by reference in their entirety as if each individual publication or patent was specifically and individually incorporated by reference. In case of conflict, the present application, including any definitions herein, may control.
de Boer, R. A., Wanner, C., Blouin, K., and Drechsler, C. (2011). Abstract 11743: Galectin-3 and Outcomes in Patients with End-Stage Renal Disease: Data from the German Diabetes and Dialysis Studyn. Circulation 124, A11743.
Dang, Z., MacKinnon, A., Marson, L. P., and Sethi, T. (2012). Tubular atrophy and interstitial fibrosis after renal transplantation is dependent on galectin-3. Transplantation 93, 477-484.
Demotte, N., Wieërs, G., Van Der Smissen, P., Moser, M., Schmidt, C., Thielemans, K., Squifflet, J.-L., Weynand, B., Carrasco, J., Lurquin, C., et al. (2010). A galectin-3 ligand corrects the impaired function of human CD4 and CD8 tumor-infiltrating lymphocytes and favors tumor rejection in mice. Cancer Res. 70, 7476-7488.
Fernandes Bertocchi, A. P., Campanhole, G., Wang, P. H. M., Gonçalves, G. M., Damião, M. J., Cenedeze, M. A., Beraldo, F. C., de Paula Antunes Teixeira, V., Dos Reis, M. A., Mazzali, M., et al. (2008). A Role for galectin-3 in renal tissue damage triggered by ischemia and reperfusion injury. Transpl. Int. 21, 999-1007.
Henderson, N. C., Mackinnon, A. C., Farnworth, S. L., Kipari, T., Haslett, C., Iredale, J. P., Liu, F.-T., Hughes, J., and Sethi, T. (2008). Galectin-3 expression and secretion links macrophages to the promotion of renal fibrosis. Am. J. Pathol. 172, 288-298.
Kolatsi-Joannou, M., Price, K. L., Winyard, P. J., and Long, D. A. (2011). Modified Citrus Pectin Reduces Galectin-3 Expression and Disease Severity in Experimental Acute Kidney Injury. PLoS ONE 6, e18683.
Leffler, H., Carlsson, S., Hedlund, M., Qian, Y., and Poirier, F. (2004). Introduction to galectins. Glycoconj. J. 19, 433-440.
Levey, A. S., Coresh, J., Greene, T., Marsh, J., Stevens, L. A., Kusek, J. W., Van Lente, F., and Chronic Kidney disease Epidemiology Collaboration (2007). Expressing the Modification of Diet in Renal Disease Study Equation for Estimating Glomerular Filtration Rate with STandardized Serum Creatinine Values. Clin Chem. 53, 766-772.
National Kidney Foundation (2002). K/DOQI Clinical Practice Guidelines for Chronic Kidney disease: Evaluation, Classification, and Stratification. Am J Kidney Dis 39, S1-S266.
Rabinovich, G. A., Toscano, M. A., Jackson, S. S., and Vasta, G. R. (2007). Functions of cell surface galectin-glycoprotein lattices. Curr. Opin. Struct. Biol. 17, 513-520.
SAS Institute Inc. SAS Language: version 8 first edition. SAS Institute, Inc, Cary, N.C., USA, 1990.
R Core Team (2012). R: A language and environment for statistical computing. R Foundation for Statistical Computing, Vienna, Austria. ISBN 3-900051-07-0, URL: http://www.R-project.org/.
Mosteller R D: Simplified Calculation of Body Surface Area. N Engl J Med 1987 Oct. 22; 317(17):1098 (letter).

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention may become apparent to those skilled in the art upon review of this specification. The full scope of the invention should be determined by reference to the claims, along with their full scope of equivalents, and the specification, along with such variations. Such equivalents are intended to be encompassed by the following claims.

1. A method for administering a galectin-3 inhibitor to a patient, comprising administering to the patient at least a first dose of a galectin-3 inhibitor, wherein the method further comprises:
a) administering the galectin-3 inhibitor weekly for an induction phase and then biweekly for a maintenance phase, wherein either administering the induction phase or administering the maintenance phase comprises administering the first dose; or
b) i) measuring a concentration, level, or activity of galectin-3 in the patient before administering the individual dose of the galectin-3 inhibitor, and
ii) measuring the concentration, level, or activity of galectin-3 after administering the individual dose of the galectin-3 inhibitor.

2-5. (canceled)

6. The method of claim 1, wherein galectin-3 inhibitor is a modified pectin.

7. The method of claim 6, wherein the backbone of the modified pectin comprises homogalacturonan and/or rhamnogalacturonan I.

8. The method of claim 6, wherein the modified pectin is de-esterified and partially depolymerized, so as to have a disrupted rhamnogalacturonan backbone.

9. The method of claim 6, wherein the modified pectin has an average molecular weight between 50 and 200 kDa, preferably between 80 and 150 kDa.

10. The method of claim 6, wherein the modified pectin is substantially free of modified pectins having molecular weights below 25 kDa.

11. The method of claim 6, wherein the modified pectin is GCS-100.

12. The method of claim 6, wherein the modified pectin is made by passing modified or unmodified pectin through a tangential flow filter.

13. The method of claim 6, comprising administering the modified pectin at a dose of about 0.1 to 2 mg/m².

14. The method of claim 13, wherein the dose is about 1.5 mg/m².

15. The method of claim 6, comprising administering the modified pectin at a dose of about 1-10 mg.

16. The method of claim 15, wherein the dose is 1, 2, 3, 4, 5, 6, 7, 8, 9, or 10 mg.

17. The method of claim 1, wherein the galectin-3 inhibitor is administered weekly or biweekly.

18. The method of claim 17, wherein the galectin-3 inhibitor is administered weekly for an induction phase and then biweekly for a maintenance phase.

19. The method of claim 18, wherein the induction phase is 1-3 months.

20. The method of claim 18, wherein the maintenance phase is at least 1 month.

21-23. (canceled)

24. The method of claim 1, wherein the at least one galectin-3 inhibitor is administered in an amount that reduces a level of galectin-3 in serum of the patient, reduces an expression level of galectin 3 in the patient, or reduces an activity of galectin-3 in the patient.

25-26. (canceled)

27. The method of claim 24, whereby the concentration, expression level, or activity of galectin-3 is reduced 0.5, 1, 2, 3, 4, or 5-fold relative to control.

28. The method of claim 1, comprising 1) measuring a concentration, level, or activity of galectin-3 in the patient before administering the individual dose of the galectin-3 inhibitor and 2) measuring the concentration, level, or activity of galectin-3 after administering the first dose of the galectin-3 inhibitor.

29. The method of claim 28, wherein a decrease in the concentration, level, or activity of galectin-3 after administering the first dose of the galectin-3 inhibitor indicates that the dose of galectin-3 inhibitor is an effective dose of galectin-3 inhibitor.

30. The method of claim 29, wherein an increase in the concentration, level, or activity of galectin-3 after administering the first dose of the galectin-3 inhibitor indicates that the dose of galectin-3 inhibitor is an ineffective dose of galectin-3 inhibitor.

31. The method of claim 30, further comprising administering to the patient a second dose of the galectin-3 inhibitor in a lower amount than the first dose.

32. The method of claim 1, further comprising administering an additional therapeutic agent.

33. The method of claim 32, wherein the additional therapeutic agent is useful for the treatment of cancer.

34. (canceled)

35. The method of claim 32, comprising administering the galectin-3 inhibitor concurrently with the additional therapeutic agent.

36. The method of claim 32, comprising administering the galectin-3 inhibitor subsequent to administration of the additional therapeutic agent.

37. The method of claim 32, comprising administering the additional therapeutic agent subsequent to administration of the galectin-3 inhibitor.

38. The method of claim 1, comprising administering multiple doses of the galectin-3 inhibitor over a period of at least 8 weeks.

39. The method of claim 1, comprising administering the galectin-3 inhibitor weekly.

40. The method of claim 1, wherein the galectin-3 inhibitor is administered orally, by injection, or by intravenous infusion.

41. (canceled)

42. The method of claim 30, further comprising increasing a dose of the induction phase or the maintenance phase that follows the individual dose.
